(19) United States
(12) Patent Application Publication
(10) Pub. No.: US 2025/0064780 A1
(43) Pub. Date: Feb. 27, 2025

(54) BRIVARACETAM TABLET AND PREPARATION METHOD THEREFOR

(71) Applicant: SUNSHINE LAKE PHARMA CO., LTD., Dongguan, Guangdong (CN)

(72) Inventors: Zhihui WANG, Dongguan (CN); Xin HUANG, Dongguan (CN); Jinsong YOU, Dongguan (CN); Fangfang HUANG, Dongguan (CN)

(73) Assignee: SUNSHINE LAKE PHARMA CO., LTD., Dongguan, Guangdong (CN)

(21) Appl. No.: 18/724,339

(22) PCT Filed: Dec. 26, 2022
(86) PCT No.: PCT/CN2022/141746

§ 371 Date: Jun. 26, 2024

(30) Foreign Application Priority Data
Dec. 27, 2021 (CN) 202111614221.4

(51) Int. Cl. A61K 31/4015; A61K 9/20; A61K 9/28

(52) U.S. Cl. CPC A61K 31/4015; A61K 9/2013; A61K 9/2018; A61K 9/2054; A61K 9/2086; A61K 9/2095; A61K 9/2893

(57) ABSTRACT

A brivaracetam tablet and a preparation method therefor, relating to the field of pharmacy. The tablet includes a sustained-release layer and an immediate-release layer; the sustained-release layer includes an active ingredient, a sustained-release framework material, and optional other pharmaceutically acceptable excipients or carriers; the immediate-release layer includes an active ingredient and other excipients or carriers; and the active ingredient is brivaracetam or a pharmaceutically acceptable salt thereof. The tablet has the advantages of fast onset time, long sustained release time, low frequency of taking, and equivalent in-vivo pharmacokinetics to a brivaracetam immediate-release tablet (reference product), and the like. The preparation method is simple to operate and stable in process.

### CROSS-REFERENCE TO RELATED APPLICATION

This is a U.S. national stage application of the International Patent Application No. PCT/CN2022/141746, filed on Dec. 26, 2022, which claims the priorities and benefits of Chinese Patent Application No. 202111614221.4, filed with the State Intellectual Property Office of China on Dec. 27, 2021, respectively, which are incorporated herein by reference in their entirety.

### FIELD OF THE INVENTION

The present invention relates to the field of pharmacy, specifically to a brivaracetam tablet and a preparation method thereof.

### BACKGROUND ART

Brivaracetam, with a chemical name of (S)-2- ((R)-2-oxo-4-propylpyrrolidin-1-yl) butanamide, is a novel synaptic vesicle protein 2A (SV2A) high-affinity ligand, as a third-generation antiepileptic drug, it can selectively bind synaptic vesicle protein 2A (SV2A), and also has a certain inhibitory effect on voltage-dependent sodium ion channels. Brivaracetam has fast oral absorption and a relatively short half-life, and the dosage form on the market is an immediate release dosage form, and there is no report on the market of extended release dosage form.

The patent application CN102046153 published a pharmaceutical composition comprising brivaracetam. The invention relates in particular to the prolonged release of a drug comprising a hydrophilic gel layer formed by the matrix in water to control the release of the drug. By wet granulation, brivaracetam was dissolved in a purified aqueous solution and sprayed into a powder bed to prepare pellets. The rate of drug release was controlled by the extended release material hydroxypropyl methylcellulose. In the process of implementing the prior art, the inventors found the following problems: because of brivaracetam is very soluble in water, pores can be formed quickly after contact with water, which will destroy the integrity of the gel layer, and it is difficult to successfully prepare hydrophilic gel skeleton tablets that can be released for a long time.

The patent application CN111407738 published a controlled-release formulation of brivaracetam and a preparation method thereof. The invention uses single-chamber osmotic pump technology and uses an elastic semi-permeable membrane control coating system to control the rate of moisture entering the tablet core. By preparing a semi-permeable film coating solution containing a film-forming material and plasticizer, the coating solution was sprayed onto the semi-permeable film coating material on the drug tablet core to form a coated tablet, and the drug release rate was controlled through the elastic semi-permeable film. In the process of implementing the prior art, the inventors found the following problems: the process was relatively complex, the equipment and process requirements for the preparation were high, and there were problems such as the slow onset time of patient administration.

After evaluation, the inventors believes that the immediate release dosage form of brivaracetam has a rapid onset of action, but the concentration of the main drug decreases quickly, and it needs to be taken twice a day, although the relevant extended release dosage form reported by the patent CN102046153 and patent CN111407738 can maintain the effective blood concentration for a long time and reduce the number of times the patient takes it, but brivaracetam as an epilepsy drug, the use of ordinary extended release dosage forms is often slow, and there are problems such as delayed onset time

The patent application WO2010089372 published a pharmaceutical composition in the form of a solid oral dosage form that uses at least one matrix agent selected from a hydrophobic matrix agent and an inert matrix. After reproducing the patent prescription, the inventors found that the technical solution had problems such as the delay in the onset time of the drug after the patient gave the drug.

Therefore, there is still an urgent need for a brivaracetam preparation with fast onset time, long sustained release time and simple preparation process.

### SUMMARY

### Summary of the Invention

In the first aspect, the present invention provides a tablet, which includes an extended release layer and an immediate release layer. The extended release layer includes an active ingredient, extended release skeleton materials and optional other pharmaceutically acceptable adjuvants or carriers, the immediate release layer includes an active ingredient and other adjuvants or carriers; the active ingredient is brivaracetam or a pharmaceutically acceptable salt thereof. The extended release skeleton material is preferably hypromellose, and more preferably is hypromellose with a solution viscosity of 13500 mPa·s-280000 mPa·s obtained by dissolving in water at 20° C.±0.1° C. with a concentration of 2 wt %. The purpose of the tablets is to solve the problems of delayed onset time, drug resistance and other problems in the extended release preparations of brivaracetam tablets in the prior art. By using tablets containing an extended release layer and an immediate release layer, the immediate release layer can quickly reach the onset of action and relieve symptoms, while the extended release layer flattens the drug and can be administered once a day to improve patient compliance and reduce toxicity. The appropriate extended release skeleton material and the appropriate prescription ratio are selected, especially the hypromellose with a viscosity of 13500 mPa·s-280000 mPa·s obtained by dissolving in water at 20° C.±0.1° C. with a concentration of 2 wt %. It is beneficial to make the accumulated release of active ingredients of the tablet in the buffer medium of pH 6.8 in 15 minutes of dissolution not less than 20% of the total mass of active ingredients, the accumulated release of active ingredients for 1 hour of dissolution is 30%-50% of the total mass of active ingredients, the accumulated release of active ingredients for 4 hours of dissolution is 50%-70% of the total mass of active ingredients, the accumulated release of active ingredients for 8 hours of dissolution is 70%-85% of the total mass of active ingredients, and the accumulated release of active ingredients for 16 hours of dissolution is not less than 90% of the total mass of active ingredients. The tablets that meet this accumulated release rule can have equivalent in vivo pharmacokinetics to the brivaracetam immediate release tablets (Reference Listed Drug) administered twice a day. Compared with the extended release tablets of the prior art, the technical solution provided in the present application greatly accelerates the onset time, and can be equivalent to the in vivo pharmacokinetics of brivaracetam immediate release tablets (Reference Listed Drug). Compared with the existing technology of brivaracetam immediate release tablets (Reference Listed Drug), the technical solution provided in this application can reduce the number of daily doses and greatly improve the patient's compliance with taking.

In the second aspect, the invention provides a method for preparing the tablet described in the first aspect. The method is simple to operate, and the process is stable.

### Details of the Invention

In order to solve the above problem, the invention provides a tablet and a method for preparing it.

In the first aspect, the invention provides a tablet.

A tablet, comprises an extended release layer and an immediate release layer, the extended release layer comprises an active ingredient, an extended release skeleton material and optionally other pharmaceutically acceptable adjuvants or carriers, the immediate release layer comprises an active ingredient and other adjuvants or carriers; the active ingredient is brivaracetam or a pharmaceutically acceptable salt thereof.

In some embodiments, a tablet comprises an extended release layer and an immediate release layer, the extended release layer comprises an active ingredient and an extended release skeleton material, the immediate release layer comprises an active ingredient and other adjuvants or carriers; and the active ingredient is brivaracetam or a pharmaceutically acceptable salt thereof.

In some embodiments, a tablet comprises an extended release layer and an immediate release layer, the extended release layer comprises an active ingredient, an extended release skeleton material and other pharmaceutically acceptable adjuvants or carriers, the immediate release layer comprises the active ingredient and other adjuvants or carriers; the active ingredient is brivaracetam or a pharmaceutically acceptable salt thereof.

In some embodiments, the extended release skeleton material comprises or is hypromellose.

In some embodiments, the viscosity of the solution obtained by dissolving the hypromellose in water at a concentration of 2 wt % at 20° C.±0.1° C. is 13500 mPa·s-280000 mPa·s.

In some embodiments, the hypromellose comprises at least one of the hypromellose K15M, hypromellose K100M and hypromellose K200M.

In some embodiments, the extended release skeleton material accounts for 30 wt %-74 wt % of the total weight of the extended release layer. In some embodiments, the extended release skeleton material accounts for 30.0 wt %-77.5 wt % of the total weight of the extended release layer. In some embodiments, the extended release skeleton material accounts for 30.0 wt %, 35.0 wt %, 40.0 wt %, 45.0 wt %, 50.0 wt %, 55.0 wt %, 60.0 wt %, 65.0 wt %, 70.0 wt %, 74.0 wt % or 77.5 wt % of the total weight of the extended release layer.

In some embodiments, the extended release skeleton material is hypromellose, the hypromellose is hypromellose K15M, the extended release skeleton material accounts for 55.5 wt %-77.5 wt % of the total weight of the extended release layer.

In some embodiments, the extended release skeleton material is hypromellose, the hypromellose is hypromellose K100M, the extended release skeleton material accounts for 46.9 wt %-77.5 wt % of the total weight of the extended release layer.

In some embodiments, the extended release skeleton material is hypromellose, the hypromellose is hypromellose K200M, the extended release skeleton material accounts for 30.0 wt %-77.5 wt % of the total weight of the extended release layer.

In some embodiments, the extended release skeleton material accounts for 22 wt %-53 wt % of the total weight of the extended release layer and the immediate release layer. In some embodiments, the extended release skeleton material accounts for 21.5 wt %-55.5 wt % of the total weight of the extended release layer and the immediate release layer. In some embodiments, the extended release skeleton material accounts for 22.0 wt %, 25.0 wt %, 30.0 wt %, 35 0 wt %, 40.0 wt %, 45 0 wt %, 50.0 wt %, 53.0 wt % or 55.5 wt % of the total weight of the extended release layer and the immediate release layer.

In some embodiments, the extended release skeleton material is hypromellose, the hypromellose is hypromellose K15M, the extended release skeleton material accounts for 39.7 wt %-55 5 wt % of the total weight of the extended release layer and the immediate release layer.

In some embodiments, the extended release skeleton material is hypromellose, the hypromellose is hypromellose K100M, the extended release skeleton material accounts for 33.6 wt %-53.5 wt % of the total weight of the extended release layer and the immediate release layer.

In some embodiments, the extended release skeleton material is hypromellose, the hypromellose is hypromellose K200M, the extended release skeleton material accounts for 21.5 wt %-55.5 wt % of the total weight of the extended release layer and the immediate release layer.

The inventors surprisingly found that when the tablet used the extended release skeleton material accounting for 30 wt %-74 wt % or 30.0 wt %-77.5 wt % of the total weight of the extended release layer and/or the extended release skeleton material accounting for 22 wt %-53 wt % or 21.5 wt %-55.5 wt % of the total weight of the extended release layer and the immediate release layer, or when the extended release skeleton material described above adopts the weight ratio of the extended release skeleton material corresponding to different types of hypromellose to the total weight of the extended release layer and/or the weight ratio of the extended release skeleton material to the total weight of the extended release layer and the immediate release layer, and when the viscosity of the solution obtained by dissolving the hypromellose in water at a concentration of 2 wt % at 20° C.±0.1° C. is 13500 mPa·s-280000 mPa·s, the tablet prepared by the present invention is intact (greater than 24 h) after swelling in water, and can avoid the rapid release of drug caused by rupture, so as to obtain an extended release preparation product with a longer extended release time and better extended release effect. On this basis, the present invention prepares a bilayer tablet of brivaracetam containing an immediate release layer and an extended release layer, the immediate release layer takes effect quickly first, and the extended release layer takes effect slowly and continuously, and the pharmacokinetic studies in the human body show that the extended release time of the immediate release and extended release double-layer tablets of brivaracetam prepared by the present invention is greater than 24 hours, which can reduce the risk caused by excessive local drug concentration, reduce the peak and valley phenomenon of blood drug concentration caused by the administration of ordinary dosage forms, and has a faster onset of effect.

In some embodiments, other pharmaceutically acceptable adjuvants or carriers in the extended release layer include at least one of diluents and lubricants

In some embodiments, other adjuvants or carriers in the immediate release layer comprise at least one of diluents, disintegrants and lubricants. In some embodiments, other adjuvants or carriers in the immediate release layer are diluents. In some embodiments, other adjuvants or carriers in the immediate release layer comprise diluents and lubricants. In some embodiments, other adjuvants or carriers in the immediate release layer comprise diluents and disintegrants. In some embodiments, other adjuvants or carriers in the immediate release layer comprise diluents, disintegrants and lubricants.

In some embodiments, the diluent in the extended release layer accounts for 0-34.0 wt % of the total mass of the extended release layer and the immediate release layer. In some embodiments, the diluent in the extended release layer accounts for 3.0 wt %, 5.0 wt %, 10.0 wt %, 15.0 wt %, 20.0 wt %, 25.0 wt %, 29.0 wt %, 30.3 wt % or 34.0 wt % of the total mass of the extended release layer and the immediate release layer.

In some embodiments, the extended release skeleton material is hypromellose, the hypromellose is hypromellose K15M, and the diluent in the extended release layer accounts for 0-15.7 wt % of the total mass of the extended release layer and the immediate release layer.

In some embodiments, the extended release skeleton material is hypromellose, the hypromellose is hypromellose K100M, and the diluent in the extended release layer accounts for 2.1 wt %-21.9 wt % of the total mass of the extended release layer and the immediate release layer.

In some embodiments, the extended release skeleton material is hypromellose, the hypromellose is hypromellose K200M, and the diluent in the extended release layer accounts for 0-34.0 wt % of the total mass of the extended release layer and the immediate release layer.

In some embodiments, the diluent in the immediate release layer accounts for 5.2 wt %-38.7 wt % of the total mass of the extended release layer and the immediate release layer. In some embodiments, the diluent in the immediate release layer accounts for 5.2 wt %, 10.0 wt %, 15.0 wt %, 20.0 wt %, 25.0 wt %, 29.0 wt %, 35.0 wt % or 38.7 wt % of the total mass of the extended release layer and the immediate release layer.

In some embodiments, the extended release skeleton material is hypromellose, the hypromellose is hypromellose K15M, and the diluent in the immediate release layer accounts for 16.5 wt %-21.7 wt % of the total mass of the extended release layer and the immediate release layer.

In some embodiments, the extended release skeleton material is hypromellose, the hypromellose is bypromellose K100M, and the diluent in the immediate release layer accounts for 21.2 wt % of the total mass of the extended release layer and the immediate release layer.

In some embodiments, the extended release skeleton material is hypromellose, the hypromellose is hypromellose K200M, and the diluent in the immediate release layer accounts for 6.2 wt %-32.9 wt % of the total mass of the extended release layer and the immediate release layer.

In some embodiments, the lubricant in the extended release layer accounts for 0-2.0 wt % of the total mass of the extended release layer and the immediate release layer. In some embodiments, the lubricant in the extended release layer accounts for 0.2 wt %-0.5 wt % of the total mass of the extended release layer and the immediate release layer. In some embodiments, the lubricant in the extended release layer accounts for 0.3 wt %-0.5 wt % of the total mass of the extended release layer and the immediate release layer. In some embodiments, the lubricant in the extended release layer accounts for 0 wt %, 1.0 wt % or 2.0 wt % of the total mass of the extended release layer and the immediate release layer.

In some embodiments, the lubricant in the immediate release layer accounts for 0-2.0 wt % of the total mass of the extended release layer and the immediate release layer. In some embodiments, the lubricant in the immediate release layer accounts for 0.2 wt %-0.5 wt % of the total mass of the extended release layer and the immediate release layer. In some embodiments, the lubricant in the immediate release layer accounts for 0.3 wt % of the total mass of the extended release layer and the immediate release layer. In some embodiments, the lubricant in the immediate release layer accounts for 0, 1.0 wt % or 2.0 wt % of the total mass of the extended release layer and the immediate release layer.

In some embodiments, the disintegrant in the immediate release layer accounts for 0-5.0 wt % of the total mass of the extended release layer and the immediate release layer. In some embodiments, the disintegrant in the immediate release layer accounts for 1.5 wt %-2.0 wt % of the total mass of the extended release layer and the immediate release layer. In some embodiments, the disintegrant in the immediate release layer accounts for 0, 1.0 wt %, 1.5 wt %, 2.0 wt %, 3.0 wt %, 4.0 wt % or 5.0 wt % of the total mass of the extended release layer and the immediate release layer.

In some embodiments, the diluent in the immediate release layer and the diluent in the extended release layer independently comprise at least one of the siliconized microcrystalline cellulose, microcrystalline cellulose, sucrose, lactose, lactose monohydrate, dicalcium phosphate, mannitol, dextrin, starch and pregelatinized starch, respectively. In some embodiments, the diluent comprises at least one of siliconized microcrystalline cellulose, microcrystalline cellulose and lactose.

In some embodiments, the lubricant in the immediate release layer and the lubricant in the extended release layer independently comprise at least one of stearic acid, tale, colloidal silicon dioxide, sodium stearic fumarate, magnesium stearate or calcium stearate, respectively. In some embodiments, the lubricant comprises at least one of stearic acid, magnesium stearate or calcium stearate.

In some embodiments, the disintegrant comprises at least one of the crospovidone, sodium starch, croscarmellose sodium, low-substituted hydroxypropyl cellulose, etc. In some embodiments, the disintegrant comprises at least one of croscarmellose sodium, etc.

In some embodiments, brivaracetam or pharmacologically acceptable salts thereof in the immediate release layer account for 16.0 wt %-33.0 wt % of the total mass of the active ingredient in the extended release layer and the immediate release layer. In some embodiments, brivaracetam or pharmacologically acceptable salts thereof in the immediate release layer account for 16.0 wt %-25.0 wt % of the total mass of the active ingredient in the extended release layer and the immediate release layer. In some embodiments, brivaracetam or pharmacologically acceptable salts thereof in the immediate release layer account for 16.0 wt %, 20.0 wt %, 25.0 wt %, 30.0 wt % or 33.0 wt % of the total mass of the active ingredient in the extended release layer and the immediate release layer.

In some embodiments, the accumulated release of active ingredients of the tablet in the buffer medium of pH6.8 in 15 minutes of dissolution not less than 20% (for example 25%, 26%, 27%, 28%, 29%, 30%, 31%, 32%, 33%, 34%, 35%, 36% or 37%) of the total mass of active ingredients, the accumulated release of active ingredients for 1 hour of dissolution is 30%-50% (for example 30%, 31%, 32%, 33%, 34%, 35%, 36%, 37%, 38%, 39%, 40%, 41%, 42%, 43%, 44%, 45%, 46%, 47%, 48%, 49% or 50%) of the total mass of active ingredients, the accumulated release of active ingredients for 4 hours of dissolution is 50%-70% (for example 50%, 51%, 52%, 53%, 54%, 55%, 56%, 57%, 58%, 59%, 60%, 61%, 62%, 63%, 64%, 65%, 66%, 67%, 68%, 69% or 70%) of the total mass of active ingredients, the accumulated release of active ingredients for 8 hours of dissolution is 70%-85% (for example 70%, 71%, 72%, 73%, 74%, 75%, 76%, 77%, 78%, 79%, 80%, 81%, 82%, 83%, 84% or 85%) of the total mass of active ingredients, and the accumulated release of active ingredients for 16 hours of dissolution is not less than 90% (for example 90%, 91%, 92%, 93%, 94%, 95%, 96%, 97%, 98%, 99% or 100%) of the total mass of active ingredients. In some embodiments, the accumulated release of active ingredients of the tablet in the buffer medium of pH 6.8 in 15 minutes of dissolution not less than 20% of the total mass of active ingredients, the accumulated release of active ingredients for 1 hour of dissolution is 30%-45% of the total mass of active ingredients, the accumulated release of active ingredients for 4 hours of dissolution is 50%-70% of the total mass of active ingredients, the accumulated release of active ingredients for 8 hours of dissolution is 70%-85% of the total mass of active ingredients, and the accumulated release of active ingredients for 16 hours of dissolution is not less than 90% of the total mass of active ingredients. Dissolution test is the use of specific methods to test the release of a drug from a formulation. It has been reported in the literatures that the dissolution test is a method that replaces the in vivo experiment by the in vitro experimental method, and the dissolution degree is closely related to the bioavailability display. Therefore, it is of great significance for pharmaceutical companies to develop an in vitro dissolution method that mimics in vivo absorption. The present invention obtains the in vivo absorption percentage-time curve by deconvolution technology according to the Wagner-Nelson method, and can establish an in vitro and in vitro correlation model (IVIVC) by drawing the in vivo drug absorption percentage-in vitro drug release percentage curve, and obtains a good linear relationship, and the correlation coefficient is greater than 0.95.

In some embodiments, the release is carried out by a paddle method.

In some embodiments, the rotational speed of the paddle method is 25 rpm-200 rpm. In some embodiments, the rotational speed of the paddle method is 50 rpm-100 rpm.

In some embodiments, the dissolution temperature of the paddle method is 37° C.±5° C.

In some embodiments, the tablet releases the active ingredient in a buffer medium at pH 6.8 for at least 24 hours.

In some embodiments, the active ingredient is calculated in brivaracetam, and the specifications of the active ingredient in the single tablet are 90 mg-120 mg. In some embodiments, the active ingredient is calculated in brivaracetam, and the specifications of the active ingredient in the single tablet are 90 mg, 95 mg, 100 mg, 115 mg or 120 mg.

In some embodiments, the tablet may also include a separator layer or coating encapsulating an extended release layer and/or an immediate release layer.

In some embodiments, the separator layer comprises a water-soluble polymer film-forming material and a plasticiser, and optionally comprises an anti-sticking agent or a sun-blocking agent. In some embodiments, the separator layer comprises a water-soluble polymer film-forming material and a plasticiser. In some embodiments, the separator layer comprises a water-soluble polymer film-forming material and a plasticiser, as well as an anti-sticking agent or a sun-blocking agent.

In some embodiments, the separator layer or coating account for 2.0 wt %-4.0 wt % of the total mass of the tablet. In some embodiments, the separator layer or coating account for 2.0 wt %, 3.0 wt% or 4.0 wt % of the total mass of the tablet.

In the second aspect, the invention provides a method for preparing the tablet described in the first aspect.

A method for preparing a tablet in the first aspect, comprising:
(1) Granulation: the active ingredient, extended release skeleton material and other pharmaceutically acceptable adjuvants or carriers are mixed, and the extended release layer particles are obtained by dry granulation; the active ingredient, other adjuvant and (2) compressing tablet: (a) extended release layer particles are pre-pressed, then filled with immediate release layer particles and tablet-compressed, or (b) immediate release layer particles are pre-pressed, then filled with extended release layer particles and tablet-compressed.

In some embodiments, the preparation method further comprises wrapping a separator layer or coating after compressing tablet.

### Beneficial Effects

Compared with the prior art, an embodiment of the above technical scheme comprises at least one of the following technical effects:
(1) The inventors surprisingly found that when the tablet used the extended release skeleton material accounting for 30 wt %-74 wt % or 30.0 wt %-77.5 wt % of the total weight of the extended release layer and/or the extended release skeleton material accounting for 22 wt %-53 wt % or 21.5 wt %-55.5 wt % of the total weight of the extended release layer and the immediate release layer, or when the extended release skeleton material described above adopts the weight ratio of the extended release skeleton material corresponding to different types of hypromellose to the total weight of the extended release layer and/or the weight ratio of the extended release skeleton material to the total weight of the extended release layer and the immediate release layer, and when the viscosity of the solution obtained by dissolving the hypromellose in water at a concentration of 2 wt % at 20° C.±0.1° C. is 13500 mPa·s-280000 mPa·s, the tablet prepared by the present invention is intact (greater than 24 h) after swelling in water, and can avoid the rapid release of drug caused by rupture, so as to obtain an extended release preparation product with a longer extended release time and better extended release effect. On this basis, the present invention prepares a bilayer tablet of brivaracetam containing an immediate release layer and an extended release layer, the immediate release layer takes effect quickly first, and the extended release layer takes effect slowly and continuously, and the pharmacokinetic studies in the human body show that the extended release time of the immediate release and extended release double-layer tablets of brivaracetam prepared by the present invention is greater than 24 hours, which can reduce the risk caused by excessive local drug concentration, reduce the peak and valley phenomenon of blood drug concentration caused by the administration of ordinary dosage forms, and has a faster onset of effect.
(2) The present invention provides an in vitro cumulative release law of brivaracetam tablets, that is, the accumulated release of active ingredients of the tablet in the buffer medium of pH 6.8 in 15 minutes of dissolution not less than 20% of the total mass of active ingredients, the accumulated release of active ingredients for 1 hour of dissolution is 30%-50% of the total mass of active ingredients, the accumulated release of active ingredients for 4 hours of dissolution is 50%-70% of the total mass of active ingredients, the accumulated release of active ingredients for 8 hours of dissolution is 70%-85% of the total mass of active ingredients, and the accumulated release of active ingredients for 16 hours of dissolution is not less than 90% of the total mass of active ingredients. The tablet provided by the present invention can satisfy the above-mentioned cumulative release law, and the tablet satisfying the cumulative release law can be equivalent to the in vivo pharmacokinetics of brivaracetam immediate release tablet (Reference Listed Drug) administered twice a day.
(3) When the tablet provided by the present invention used the extended release skeleton material accounting for 30 wt %-77.5 wt % of the total weight of the extended release layer and/or the extended release skeleton material accounting for 21.5 wt %-55.5 wt % of the total weight of the extended release layer and the immediate release layer, or when the extended release skeleton material described above adopts the weight ratio of the extended release skeleton material corresponding to different types of hypromellose to the total weight of the extended release layer and/or the weight ratio of the extended release skeleton material to the total weight of the extended release layer and the immediate release layer, and when the viscosity of the solution obtained by dissolving the hypromellose in water at a concentration of 2 wt % at 20° C.±0.1° C. is 13500 mPa·s-280000 mPa·s, and when brivaracetam or pharmacologically acceptable salt thereof in the immediate release layer accounts for 16.0 wt %-33.0 wt % or 16.0 wt %-25.0 wt % of the total mass of the active ingredient in the extended release layer and the immediate release layer, the tablet can satisfy the cumulative release law of the above-mentioned brivaracetam tablets in vitro.
(4) Compared with the extended release tablets of the prior art, the technical scheme provided by the present invention greatly accelerates the onset time, and can be equivalent to the in vivo pharmacokinetics of brivaracetam immediate release tablets (Reference Listed Drug). Compared with the brivaracetam immediate release tablets (Reference Listed Drug) of the prior art, the technical scheme provided by the present invention can reduce the number of daily doses and greatly improve the patient's compliance with taking.
(5) In vitro and in vivo data show that after oral administration, the tablet prepared by the present invention can be partially rapidly released, part of the continuous release, the blood drug concentration is kept in a relatively stable and lasting effective range, the sustained release effect in the human body reaches 24 hours, the peak and valley phenomenon of blood drug concentration presented by the administration of ordinary dosage form is reduced, it has the characteristics of rapid onset, less number of takes, convenient carrying, convenient transportation, good stability, improving the patient's drug compliance, improving the safety of the drug, and the preparation process is simple, and has the characteristics of broad application prospects.

### Explanation of Term

As used in the present invention, the following words and phrases are generally intended to have the meanings set forth below, unless the context otherwise indicates:
The term “AUC” indicates the area enclosed by the plasma concentration curve against the timeline.
The term “AUC₀₋ₜ” or “AUCₗₐₛₜ” indicates the area under the drug concentration-time curve from 0 to the sample collection time t at which the last concentration can be accurately determined.
The term “AUC_{0-inf}” or “AUC_{0-∞}” indicates the area under the drug concentration-time curve from 0 to infinite time (∞)
The term “Tₘₐₓ” indicates the time required to reach the peak concentration of the drug after administration.
The term “Cₘₐₓ” indicates the highest blood concentration that occurs after administration.
The term “ER” indicates the extended release layer.
The term “IR” indicates the immediate release layer.
The term “comprise” or “include” and variations thereof such as “comprises” and “comprising” or “includes” and “including” should be understood as open-ended, i.e. “comprise but not limited to”. When used to define compositions and methods, “basically consists of . . . ” or its grammatical variants shall indicate the exclusion of other elements of any importance to the composition and the method of preparation, but not factors that have no substantial effect on the composition and the method of preparation. “Consists of . . . ” or its grammatical variants shall indicate the exclusion of elements not explicitly enumerated. The embodiments defined by each of these transitional terms are within the scope of the present invention. For example, when a formulation is described as comprising components A, B, and C, the formulation is essentially composed of A, B, and C, and the formulation consists of A, B, and C, independently within the scope of the present invention.

The singular forms “a” “an” and “the” comprise the plurals unless the context clearly dictates otherwise. For example, reference to “excipient” comprises more excipients.

Unless the context clearly dictates otherwise, “more” or “plurality” comprises plural references of two and more. For example, “more excipients” comprises two or more excipients.

As used herein, the term “about” in the context of a quantitative measurement refers to ±10%, ±5% or ≡1% of the stated value. For example, “about 10” means 9-11, 9.5-10.5 or 9.9-10.1. The term “about X” also comprises “X”

The descriptions of numerical ranges herein are intended to be used as a shorthand method of individually referring to each individual value falling within that range. Unless otherwise indicated herein, each individual value is incorporated into this specification as if individually referenced herein.

The term “wt %” as used herein refers to the weight of a component based on the total weight of the composition containing that component.

The term “pharmaceutically acceptable” refers to a material that is not biologically or otherwise undesirable, e.g., which material may be incorporated into a pharmaceutical formulation administered to a patient without causing any significant adverse biological effects or in any way interacts with other components of the formula in harmful ways. Pharmaceutically acceptable carriers (eg, carriers, adjuvants and/or other excipients) preferably meet toxicological and manufacturing testing standards and/or contain inactive ingredients within the guidelines established by the United States Food and Drug Administration.

The term “adjuvants” or “excipients”, or “pharmaceutically acceptable adjuvants” or “pharmaceutically acceptable excipients”, refers to fillers, diluents, disintegrants, precipitates, etc. that are administered with the compound Inhibitors, surfactants, glidants, binders, lubricants and other excipients and carriers. The adjuvants or excipients herein are described in “Remington's Pharmaceutical Sciences” by E. W. Martin.

### DESCRIPTION OF THE DRAWINGS

FIG. 1 is a graph showing the blood drug concentration-time curves of tablet F1 prescription, tablet F3 prescription and reference listed drug in beagle dogs in Comparative Example 1.

FIG. 2 is a graph showing the blood drug concentration-time curves of tablet F4 prescription and reference listed drug in beagle dogs in Comparative Example 1.

FIG. 3 is a blood concentration-time curve diagram of the T1 prescription (tablet F23) and RLD (reference listed drug) of Example 15.

FIG. 4 is a dissolution amount-cumulative absorption chart of the T1 prescription (tablet F23) in Example 15.

FIG. 5 is a comparison chart between the model simulation prediction data and the actual data of the extended release part of the T1 prescription (tablet F23) in Example 16.

FIG. 6 is a comparison chart between the model simulation prediction data of the blood drug concentration-time curve of the F1 prescription and the actual data of the blood drug concentration-time curve of the T1 prescription and RLD in Example 17.

FIG. 7 is a comparison chart between the model simulation prediction data of the blood drug concentration-time curve of the F1 prescription and the actual data of the blood drug concentration-time curve of the T1 prescription and RLD in Example 17.

FIG. 8 is a comparison chart between the model simulation prediction data of the blood drug concentration-time curve of the F1 prescription and the actual data of the blood drug concentration-time curve of the T1 prescription and RLD in Example 17.

FIG. 9 is a comparison chart between the model simulation prediction data of the blood drug concentration-time curve of the F3 prescription and the actual data of the blood drug concentration-time curve of the T1 prescription and RLD in Example 18.

FIG. 10 is a comparison chart between the model simulation prediction data of the blood drug concentration-time curve of the F3 prescription and the actual data of the blood drug concentration-time curve of the T1 prescription and RLD in Example 18.

FIG. 11 is a comparison chart between the model simulation prediction data of the blood drug concentration-time curve of the F6 prescription and the actual data of the blood drug concentration-time curve of the T1 prescription in Example 19.

FIG. 12 is a comparison chart between the model simulation prediction data of the blood drug concentration-time curve of the F6 prescription and the actual data of the blood drug concentration-time curve of the T1 prescription and RLD in Example 19.

FIG. 13 is a comparison chart between the model simulation prediction data of the blood drug concentration-time curve of the F4 prescription and the actual data of the blood drug concentration-time curve of the T1 prescription in Example 20.

FIG. 14 is a comparison chart between the model simulation prediction data of the blood drug concentration-time curve of the F4 prescription and the actual data of the blood drug concentration-time curve of the T1 prescription and RLD in Example 20.

FIG. 15 is a comparison chart between the model simulation prediction data of the blood drug concentration-time curve of the F4 prescription and the actual data of the blood drug concentration-time curve of the T1 prescription and RLD in Example 20.

FIG. 16 is a comparison chart between the model simulation prediction data of the blood drug concentration-time curve of the F7 prescription and the actual data of the blood drug concentration-time curve of the T1 prescription and RLD in Example 21.

FIG. 17 is a comparison chart between the model simulation prediction data of the blood drug concentration-time curve of the F7 prescription and the actual data of the blood drug concentration-time curve of the T1 prescription and RLD in Example 21.

FIG. 18 is a comparison chart between the model simulation prediction data of the blood drug concentration-time curve of the F13 prescription and the actual data of the blood drug concentration-time curve of the T1 prescription and RLD in Example 22.

FIG. 19 is a comparison chart between the model simulation prediction data of the blood drug concentration-time curve of the F13 prescription and the actual data of the blood drug concentration-time curve of the T1 prescription and RLD in Example 22.

FIG. 20 is a comparison chart between the model simulation prediction data of the blood drug concentration-time curve of the F13 prescription and the actual data of the blood drug concentration-time curve of the T1 prescription and RLD in Example 22.

FIG. 21 is a comparison chart between the model simulation prediction data of the blood drug concentration-time curve of the F15 prescription and the actual data of the blood drug concentration-time curve of the T1 prescription and RLD in Example 23.

FIG. 22 is a comparison chart between the model simulation prediction data of the blood drug concentration-time curve of the F15 prescription and the actual data of the blood drug concentration-time curve of the T1 prescription and RLD in Example 23.

FIG. 23 is a comparison chart between the model simulation prediction data of the blood drug concentration-time curve of the F15 prescription and the actual data of the blood drug concentration-time curve of the T1 prescription and RLD in Example 23.

FIG. 24 is a comparison chart between the model simulation prediction data of the blood drug concentration-time curve of the F19 prescription and the actual data of the blood drug concentration-time curve of the T1 prescription and RLD in Example 24.

FIG. 25 is a comparison chart between the model simulation prediction data of the blood drug concentration-time curve of the F19 prescription and the actual data of the blood drug concentration-time curve of the T1 prescription and RLD in Example 24

FIG. 26 is a comparison chart between the model simulation prediction data of the blood drug concentration-time curve of the F19 prescription and the actual data of the blood drug concentration-time curve of the T1 prescription and RLD in Example 24.

FIG. 27 is a comparison chart between the model simulation prediction data of the blood drug concentration-time curve of the F43 prescription and the actual data of the blood drug concentration-time curve of the T1 prescription and RLD in Example 25.

FIG. 28 is a comparison chart between the model simulation prediction data of the blood drug concentration-time curve of the F43 prescription and the actual data of the blood drug concentration-time curve of the T1 prescription and RLD in Example 25.

FIG. 29 is a comparison chart between the model simulation prediction data of the blood drug concentration-time curve of the F43 prescription and the actual data of the blood drug concentration-time curve of the T1 prescription and RLD in Example 25.

FIG. 30 is a comparison chart between the model simulation prediction data of the blood drug concentration-time curve of the F44 prescription and the actual data of the blood drug concentration-time curve of the T1 prescription and RLD in Example 26.

FIG. 31 is a comparison chart between the model simulation prediction data of the blood drug concentration-time curve of the F44 prescription and the actual data of the blood drug concentration-time curve of the T1 prescription and RLD in Example 26.

FIG. 32 is a comparison chart between the model simulation prediction data of the blood drug concentration-time curve of the F44 prescription and the actual data of the blood drug concentration-time curve of the T1 prescription and RLD in Example 26.

FIG. 33 is a comparison chart between the model simulation prediction data of the blood drug concentration-time curve of the F46 prescription and the actual data of the blood drug concentration-time curve of the T1 prescription and RLD in Example 27.

FIG. 34 is a comparison chart between the model simulation prediction data of the blood drug concentration-time curve of the F46 prescription and the actual data of the blood drug concentration-time curve of the T1 prescription and RLD in Example 27.

FIG. 35 is a comparison chart between the model simulation prediction data of the blood drug concentration-time curve of the F46 prescription and the actual data of the blood drug concentration-time curve of the T1 prescription and RLD in Example 27.

FIG. 36 is a comparison chart between the model simulation prediction data of the blood drug concentration-time curve of the F47 prescription and the actual data of the blood drug concentration-time curve of the T1 prescription and RLD in Example 28.

FIG. 37 is a comparison chart between the model simulation prediction data of the blood drug concentration-time curve of the F47 prescription and the actual data of the blood drug concentration-time curve of the T1 prescription and RLD in Example 28.

FIG. 38 is a comparison chart between the model simulation prediction data of the blood drug concentration-time curve of the F47 prescription and the actual data of the blood drug concentration-time curve of the T1 prescription and RLD in Example 28.

FIG. 39 is a comparison chart between the model simulation prediction data of the blood drug concentration-time curve of the F21 prescription and the actual data of the blood drug concentration-time curve of the T1 prescription and RLD in Example 29.

FIG. 40 is a comparison chart between the model simulation prediction data of the blood drug concentration-time curve of the F21 prescription and the actual data of the blood drug concentration-time curve of the T1 prescription and RLD in Example 29.

FIG. 41 is a comparison chart between the model simulation prediction data of the blood drug concentration-time curve of the F21 prescription and the actual data of the blood drug concentration-time curve of the T1 prescription and RLD in Example 29.

### EXAMPLES

Those skilled in the art will understand that the following examples are intended to illustrate the invention and are not to be construed as limiting the invention. Various modifications and variations of the present invention may occur to those skilled in the art. Unless otherwise specified, if specific techniques or conditions are not explicitly described in the following embodiments, those skilled in the art can proceed according to commonly used techniques or conditions in the field or according to product instructions. If the manufacturer of the drugs, reagents or instruments used is not indicated, they are all conventional products that can be purchased on the market.

In the following examples, the term “additional” means adding and mixing after the granulation of the immediate release layer or extended release layer is completed.

Unless otherwise stated, the sources of reagents used in the following examples are:
Hypromellose K100LV (trade name: Benecel™ K100LV PH PRM), hypromellose K4M (trade name: Benecel™ K4M Pharm), hypromellose K15M (trade name: Benecel™ K15M Pharm), hypromellose K200M (trade name: Benecel™ K200M Pharm), polyethylene oxide (trade name: POLYOX WSR 60K)

In the following embodiments, in order to simplify the length, individual reagents are referred to by abbreviations, for example: hypromellose K100LV is referred to as K100LV, hypromellose K4M is referred to as K4M, hypromellose K15M is referred to as K15M, hypromellose K100M is referred to as K100M, hypromellose K200M is referred to as K200M, and polyethylene oxide is referred to as PEO.

In the following embodiment, the viscosity of the solution obtained by dissolving the different hypromellose models in water at a concentration of 2 wt % at 20° C.±0.1° C. is: the viscosity of K100LV is 100 mPa·s, the viscosity of K4M is 2700˜5040 mPa·s, the viscosity of K15M is 13500˜25200 mPa·s, the viscosity of K100M is 75000˜140000 mPa·s, and the viscosity of K200M is 150000˜280000 mPa·s.

### Comparative Example 1: Preparation of 100 mg of Brivaracetam Extended-Release Tablets

| Ingredient | | F1/mg | F2/mg | F3/mg | F4/mg |
|---|---|---|---|---|---|
| Brivaracetam | | 100.00 | 100.00 | 100.00 | 100.00 |
| Extended release | Hypromellose K15M | 180.00 | 270.00 | 450.00 | / |
| skeleton material | Hypromellose K100LV | / | / | / | 120.00 |

| | | | | |
|---|---|---|---|---|
| Lactose | 207.40 | 147.40 | 27.40 | 235.00 |
| Microcrystalline cellulose | 103.60 | 73.60 | 13.60 | 136.00 |
| Colloidal silica | 3.00 | 3.00 | 3.00 | 3.00 |
| Magnesium stearate | 6.00 | 6.00 | 6.00 | 6.00 |

Preparation method: according to the prescription described in the comparative example 1 (the prescription refers to the patent CN102046153 prescription), 1) the 20-mesh brivaracetam was mixed with extended release skeleton material, lactose, and microcrystalline cellulose for 30 min, and then the sieved colloidal silica and magnesium stearate were added to the mixing bucket and the mixture was mixed for 5 min, and the particles were pressed into extended release tablets; 2) opadry 85F coating solution was configured to coat the above-mentioned extended release tablet core, and the percentage of coating weight gain in the tablet core was 2%.

According to USP<711>, the volume of the medium was 900±9 ml, the temperature of the medium was 37.0±0.5° C., and the rotational speed of the paddle method was 50 rpm or 100 rpm. The dissolution characteristics of tablets F1, F2, F3 and F4 in vitro pH 6.8 dissolution medium were determined at 16 h intervals.

TABLE 1
Dissolution (%) (n = 3) of comparative example 1 and reference listed drug in pH 6.8 medium

| | | Weight of | | | | | | | | |
|---|---|---|---|---|---|---|---|---|---|---|
| | Types of | extended release | | | | | | | | |
| Group - | extended | material to the | | | | | | | | |
| Dissolution | release | total weight of | | | | | | | | |
| speed | material | table core | 15 min | 30 min | 1 h | 2 h | 4 h | 8 h | 12 h | 16 h |
| F1 -50 rpm | Hypromellose | 30.0% | 11 | 19 | 31 | 48 | 70 | 90 | 97 | 98 |
| F1 -100 rpm | K15M | | 12 | 21 | 33 | 53 | 75 | 96 | 100 | 100 |
| F2 -50 rpm | Hypromellose | 45.0% | 11 | 18 | 27 | 42 | 60 | 82 | 92 | 97 |
| F2 -100 rpm | K15M | | 14 | 21 | 29 | 44 | 61 | 82 | 92 | 98 |
| F3 -50 rpm | Hypromellose | 75.0% | 10 | 15 | 23 | 34 | 49 | 69 | 81 | 89 |
| F3 -100 rpm | K15M | | 13 | 18 | 25 | 36 | 50 | 69 | 81 | 92 |
| F4 -50 rpm | Hypromellose | 20.0% | 24 | 39 | 63 | 86 | 94 | 97 | 98 | 98 |
| F4 -100 rpm | K100LV | | 28 | 47 | 76 | 94 | 100 | 100 | 100 | 100 |
| Reference listed | / | / | 101 | — | — | — | — | — | — | — |
| drug (trade | | | | | | | | | | |
| name: | | | | | | | | | | |
| BRIVIACT) | | | | | | | | | | |

Tablets F1, F2, F3 and F4 were prepared according to the prescription described in comparative example 1 (the prescription refers to the patent CN102046153 prescription), and the cumulative release in 15 minutes of tablets F1, tablets F2 and tablets F3 were less than 20%. Tablet F4 was replaced with low-viscosity hydroxypropyl methyl cellulose K100LV, and the cumulative release in 15 min was greater than 20%, but the tablet was dissolved too quickly and the sustained release time was too short. The dissolution data of tablets F1, F2, F3 and F4 show that the tablets prepared according to the prescription described in comparative example 1 (the prescription refers to the patent CN102046153 prescription) do not well meet the requirements of the patent for the dissolution release (the mass of the active ingredient released cumulatively in a buffer medium at pH 6.8 is not less than 20% of the total active ingredient mass at 15 minutes of dissolution, the mass of the active ingredient cumulatively released at 1 hour of dissolution is 30%-50% of the total mass of the active ingredient, the mass of the active ingredient cumulatively released at 4 hours of dissolution is 50%-70% of the total mass of the active ingredient, and the mass of the active ingredient cumulatively released after 8 hours of dissolution is 70%-85% of the total mass of the active ingredient, and the mass of the active ingredient cumulatively released 16 hours of dissolution is not less than 90% of the total mass of the active ingredient, which can be continuously released for 24 hours).

### Comparative Example 2: Preparation of 100 mg of Brivaracetam Extended-Release Tablets

| | |
|---|---|
| Ingredient | F5/mg |

Prescription:

| | |
|---|---|
| Brivaracetam | 100.00 |
| Hypromellose K4M | 45.00 |
| Sorbitol | 12.00 |
| Microcrystalline cellulose | 40.00 |
| Colloidal silica | 1.00 |
| Magnesium stearate | 2.00 |

Coating

| | |
|---|---|
| Eudragit RL 30D | 5.61 |
| Eudragit RS 30 | 50.46 |
| Triethyl citrate | 3.33 |
| Talcum powder | 8.42 |
| Water | 75.03 |

Preparation method: according to the prescription described in the comparative example 2 (the prescription refers to the patent CN111407738 prescription), 1) after pre-mixing hypromellose K4M as a hydrophilic matrix with sorbitol and microcrystalline cellulose for 5 min, brivaracetam with a 20-mesh sieve was added and mixed for 5 min, and then colloidal silica and magnesium stearate were added sequentially, the mixture was mixed for 2 min and 1 min respectively, and the tablet was pressed to obtain the tablet core; 2) Eudragit RL30D/RS30D coating solution was configured to coat the above-mentioned extended release tablet core.

Tablet F6 adopts the prescription process in tablet F5, holes were punched on the side of the short axis and the side of the long axis of the coating special-shaped piece (12.6*5.4 mm), with an aperture of 0.6 mm.

According to USP<711> the volume of the medium was 900±9 ml, the temperature of the medium was 37.0±0.5° C., and the rotational speed of the paddle method was 50 rpm or 100 rpm. The dissolution characteristics of tablets F5 and F6 in vitro pH 6.8 dissolution medium were determined at 16 h intervals.

TABLE 2
Dissolution (%) (n = 3) of comparative example
2 and reference listed drug in pH 6.8 medium

| Group - | | | | | | | | |
|---|---|---|---|---|---|---|---|---|
| Dissolution | | | | | | | | |
| speed | 15 min | 30 min | 1 h | 2 h | 4 h | 8 h | 12 h | 16 h |
| F5 -50 rpm | 3 | 4 | 7 | 14 | 32 | 51 | 76 | 95 |
| F5 -100 rpm | 4 | 5 | 9 | 16 | 31 | 55 | 78 | 97 |
| F6 - long axis | 3 | 6 | 9 | 14 | 28 | 53 | 79 | 97 |
| side - 50 rpm | | | | | | | | |
| F6 - long axis | 6 | 8 | 12 | 19 | 33 | 55 | 81 | 99 |
| side - 100 rpm | | | | | | | | |
| Reference listed | 101 | — | — | — | — | — | — | — |
| drug (trade | | | | | | | | |
| name: | | | | | | | | |
| BRIVIACT) | | | | | | | | |

Tablets F5 and F6 were prepared according to the prescription described in comparative example 2 (the prescription refers to the patent CN111407738 prescription), and the dissolution of brivaracetam extended-release tablets was too slow, and the dissolution data could not well meet the requirements of the patent for the dissolution release amount (the mass of the active ingredient released cumulatively in a buffer medium at pH 6.8 is not less than 20% of the total active ingredient mass at 15 minutes of dissolution, the mass of the active ingredient cumulatively released at 1 hour of dissolution is 30%-50% of the total mass of the active ingredient, the mass of the active ingredient cumulatively released at 4 hours of dissolution is 50%-70% of the total mass of the active ingredient, and the mass of the active ingredient cumulatively released after 8 hours of dissolution is 70%-85% of the total mass of the active ingredient, and the mass of the active ingredient cumulatively released 16 hours of dissolution is not less than 90% of the total mass of the active ingredient, which can be continuously released for 24 hours).

### Comparative Example 3: Preparation of 100 mg of Brivaracetam Extended-Release Tablets

| Ingredient | F7/mg |
|---|---|
| Brivaracetam | 100.00 |
| Glyceryl distearate | 494.00 |
| Microcrystalline cellulose | 73.60 |
| Lactose monohydrate | 147.40 |
| Colloidal silica | 3.00 |
| Magnesium stearate | 6.00 |

Preparation method: according to the prescription described in comparative example 3 (the prescription refers to the patent WO2010089372 prescription), 1) brivaracetam through a 20-mesh sieve, glyceryl distearate, microcrystalline cellulose and lactose monohydrate were mixed for 30 min, and then colloidal silica and magnesium stearate were added sequentially, the mixture was mixed for 2 min and 1 min respectively, and the tablet was pressed to obtain the tablet core; 2) Opadry 85F coating solution was configured to coat the above-mentioned extended-release tablet core, and the percentage of coating weight gain in the tablet core was 2%.

According to USP<711> the volume of the medium was 900±9ml, the temperature of the medium was 37.0±0.5° C., and the rotational speed of the paddle method was 50 rpm or 100 rpm. The dissolution characteristics of tablet F7 in vitro pH 6.8 dissolution medium were determined at 16 h intervals.

TABLE 3
Dissolution (%) (n = 3) of comparative example 3 and reference listed drug in pH 6.8 medium

| | Types of | Weight of extended | | | | | | | | |
|---|---|---|---|---|---|---|---|---|---|---|
| Group - | extended | release material to | | | | | | | | |
| Dissolution | release | the total weight of | | | | | | | | |
| speed | material | table core | 15 min | 30 min | 1 h | 2 h | 4 h | 8 h | 12 h | 16 h |
| F7-50 rpm | Glyceryl | 60.0% | 1 | 7 | 21 | 47 | 78 | 93 | 97 | 100 |
| F7-100 rpm | distearate | | 2 | 9 | 25 | 52 | 83 | 99 | 100 | 100 |
| Reference | / | / | 101 | — | — | — | — | — | — | — |
| listed drug | | | | | | | | | | |
| (trade name: | | | | | | | | | | |
| BRIVIACT) | | | | | | | | | | |

In the preparation of tablet F7 according to the prescription described in comparative example 3 (the prescription refers to the patent WO2010089372 prescription), the dissolution of brivaracetam extended release tablets was too slow, and the dissolution data could not well meet the requirements of the patent for the dissolution release amount (the mass of the active ingredient released cumulatively in a buffer medium at pH 6.8 is not less than 20% of the total active ingredient mass at 15 minutes of dissolution, the mass of the active ingredient cumulatively released at 1 hour of dissolution is 30%-50% of the total mass of the active ingredient, the mass of the active ingredient cumulatively released at 4 hours of dissolution is 50%-70% of the total mass of the active ingredient, and the mass of the active ingredient cumulatively released after 8 hours of dissolution is 70%-85% of the total mass of the active ingredient, and the mass of the active ingredient cumulatively released 16 hours of dissolution is not less than 90% of the total mass of the active ingredient, which can be continuously released for 24 hours).

Comprehensively for the comparative example 1, the comparative example 2 and the comparative example 3, the brivaracetam extended-release tablets prepared by the existing published patents CN102046153, CN111407738 and WO2010089372 can not well meet the requirements of this patent for the amount of dissolution release, that is, the mass of the active ingredient released cumulatively in a buffer medium at pH 6.8 is not less than 20% of the total active ingredient mass at 15 minutes of dissolution, the mass of the active ingredient cumulatively released at 1 hour of dissolution is 30%-50% of the total mass of the active ingredient, the mass of the active ingredient cumulatively released at 4 hours of dissolution is 50%-70% of the total mass of the active ingredient, and the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient, which can be continuously released for 24 hours. The brivaracetam extended-release tablets prepared for comparative example 1, comparative example 2 and comparative example 3 all showed slow release in the front, or fast release in the front and faster release in the back, and the dissolution was unstable.

### Example 1: Preparation of 100 mg Brivaracetam Immediate-Release Tablets

| | | | | |
|---|---|---|---|---|
| Ingredient | F8/mg | F9/mg | F10/mg | F11/mg |

Inner phase:

| | | | | |
|---|---|---|---|---|
| Brivaracetam | 100.00 | 100.00 | 100.00 | 100.00 |
| Lactose monohydrate | 205.10 | 197.10 | 205.10 | / |
| Microcrystalline | / | / | / | 205.10 |
| cellulose | | | | |
| Croscarmellose Sodium | 16.20 | 16.20 | 8.10 | 16.20 |
| Magnesium stearate | 2.70 | 2.70 | 2.70 | 2.70 |

Outer phase:

| | | | | |
|---|---|---|---|---|
| Anhydrous lactose | 197.10 | 205.10 | 197.10 | / |
| Microcrystalline | / | / | / | 197.10 |
| cellulose | | | | |
| Croscarmellose Sodium | 16.20 | 16.20 | 24.30 | 16.20 |
| Magnesium stearate | 2.70 | 2.70 | 2.70 | 2.70 |

Preparation method: 1) brivaracetam through a 20-mesh sieve was mixed with lactose monohydrate and croscarmellose sodium for 30 min, magnesium stearate was added to continue mixing for 5 min, and the mixture was then dried granulation; 2) the sized granules were mixed with anhydrous lactose and croscarmellose sodium for 30 min, magnesium stearate was added to the mixing bucket and the mixture was continued to mix for 5 min, as an immediate release layer particle, and the tablet was pressed to obtain the tablet core; 3) Opadry 85F coating solution was configured to coat the above-mentioned immediate release tablet core, and the percentage of coating weight gain in the tablet core was 2%.

According to USP<711> the volume of the medium was 900±9ml, the temperature of the medium was 37.0±0.5° C., and the rotational speed of the paddle method was 50 rpm or 100 rpm. The dissolution characteristics of tablets F8, F9, F10 and F11 in vitro pH 6.8 dissolution medium were determined at 16 h intervals.

TABLE 4
Dissolution (%) (n = 3) of example 1 and reference listed drug in pH 6.8 medium

| Group - Dissolution speed | 5 min | 10 min | 15 min | 20 min | 30 min | 60 min |
|---|---|---|---|---|---|---|
| F8-50 rpm | 95 | 97 | 97 | 98 | 98 | 98 |
| F8-100 rpm | 96 | 98 | 99 | 99 | 99 | 99 |
| F9-50 rpm | 94 | 97 | 98 | 98 | 98 | 98 |
| F9-100 rpm | 95 | 99 | 99 | 99 | 100 | 100 |
| F10-50 rpm | 95 | 96 | 98 | 98 | 98 | 98 |
| F10-100 rpm | 98 | 99 | 99 | 99 | 99 | 100 |
| F11-50 rpm | 95 | 97 | 98 | 98 | 98 | 98 |
| F11-100 rpm | 98 | 100 | 100 | 100 | 100 | 100 |
| Reference listed drug | 57 | 92 | 100 | 101 | 101 | 101 |
| (trade name: BRIVIACT) | | | | | | |

In example 1, the prescription of high-specification 100 mg brivaracetam was investigated, and the dissolution result showed that the solubility of brivaracetam was good, and the type and dosage of the immediate release layer prescription had little influence on the dissolution. Based on this, the prescription of the immediate release layer was no longer investigated in other examples of the invention.

### Example 2: Preparation of 100 mg Brivaracetam Immediate Release Extended Release Bilayer Tablets

| | | | | | | | |
|---|---|---|---|---|---|---|---|
| Ingredient | F12/mg | F13/mg | F14/mg | F15/mg | F16/mg | F17/mg | F18/mg |

Immediate release layer:

| | | | | | | | |
|---|---|---|---|---|---|---|---|
| Brivaracetam | 25.00 | 25.00 | 25.00 | 25.00 | 25.00 | 25.00 | 25.00 |
| Lactose monohydrate | 51.26 | 51.26 | 51.26 | 51.26 | 51.26 | 51.26 | 51.26 |
| Croscarmellose Sodium | 4.05 | 4.05 | 4.05 | 4.05 | 4.05 | 4.05 | 4.05 |
| Magnesium stearate | 0.68 | 0.68 | 0.68 | 0.68 | 0.68 | 0.68 | 0.68 |
| Anhydrous lactose | 49.28 | 49.28 | 49.28 | 49.28 | 49.28 | 49.28 | 49.28 |
| (additional) | | | | | | | |
| Croscarmellose Sodium | 4.05 | 4.05 | 4.05 | 4.05 | 4.05 | 4.05 | 4.05 |
| (additional) | | | | | | | |
| Magnesium stearate | 0.68 | 0.68 | 0.68 | 0.68 | 0.68 | 0.68 | 0.68 |
| (additional) | | | | | | | |

Extended release layer:

| Brivaracetam | | 75.00 | 75.00 | 75.00 | 75.00 | 75.00 | 75.00 | 75.00 |
|---|---|---|---|---|---|---|---|---|
| Extended | Hypromellose | 102.00 | / | / | / | / | / | / |
| release | K100LV | | | | | | | |
| skeleton | Hypromellose | / | 102.00 | / | / | / | / | / |
| material | K4M | | | | | | | |
| | Hypromellose | / | / | 102.00 | / | / | / | / |
| | K15M | | | | | | | |
| | Hypromellose | / | / | / | 102.00 | / | / | / |
| | K100M | | | | | | | |
| | Hypromellose | / | / | / | / | 102.00 | / | / |
| | K200M | | | | | | | |
| | Polyethylene | / | / | / | / | / | 102.00 | / |
| | oxide (PEO) | | | | | | | |
| | Tribehenin | / | / | / | / | / | / | 102.00 |

| | | | | | | | |
|---|---|---|---|---|---|---|---|
| Magnesium stearate | 0.79 | 0.79 | 0.79 | 0.79 | 0.79 | 0.79 | 0.79 |
| Silicified microcrystalline | 161.42 | 161.42 | 161.42 | 161.42 | 161.42 | 161.42 | 161.42 |
| cellulose (additional) | | | | | | | |
| Magnesium stearate | 0.79 | 0.79 | 0.79 | 0.79 | 0.79 | 0.79 | 0.79 |
| (additional) | | | | | | | |

Preparation method:
1. Extended release layer:
   1) Brivaracetam through a 20-mesh sieve was mixed with the extended release skeleton material for 30 min, and the magnesium stearate was added to the mixing bucket for 5 min, and the mixture was then dried granulation;
   2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer:
   1) Brivaracetam through a 20-mesh sieve was mixed with the lactose monohydrate and croscarmellose sodium for 30 min, and the magnesium stearate was added to the mixing bucket for 5 min, and the mixture was then dried granulation;
   2) the sized granules were mixed with anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the particles were pressed into a bilayer tablet;
4. Coating: Opadry 85F coating solution was configured to coat the above-mentioned immediate release extended release bilayer tablet core, and the percentage of coating weight gain in the tablet core was 2%.

Dissolution detection: According to USP<711>, the volume of the medium was 900±9ml, the temperature of the medium was 37.010.5° C., and the rotational speed of the paddle method was 50 rpm or 100 rpm. The dissolution characteristics of tablets F12, F13, F14, F15, F16, F17 and F18 in vitro pH 6.8 dissolution medium were determined at 16 h intervals, the results are shown in Table 5.

TABLE 5
Dissolution (%) (n = 3) of example 2 and reference listed drug in pH 6.8 medium

| | | Weight of | | | | | | | | | |
|---|---|---|---|---|---|---|---|---|---|---|---|
| | | extended | Weight of | | | | | | | | |
| | | release | extended release | | | | | | | | |
| | Types of | material to | material to the | | | | | | | | |
| Group - | extended | the total | total weight of | | | | | | | | |
| Dissolution | release | weight of | extended release | | | | | | | | |
| speed | material | table core | layer | 15 min | 30 min | 1 h | 2 h | 4 h | 8 h | 12 h | 16 h |
| F12-50 rpm | Hypromellose | 21.5% | 30.0% | 43 | 54 | 72 | 89 | 95 | 97 | 98 | 98 |
| F12-100 rpm | K100LV | | | 47 | 62 | 85 | 94 | 100 | 100 | 100 | 100 |
| F13-50 rpm | Hypromellose | 21.5% | 30.0% | 40 | 51 | 69 | 84 | 91 | 97 | 100 | 100 |
| F13-100 rpm | K4M | | | 45 | 58 | 80 | 93 | 99 | 99 | 99 | 99 |
| F14-50 rpm | Hypromellose | 21.5% | 30.0% | 33 | 45 | 56 | 68 | 78 | 94 | 99 | 99 |
| F14-100 rpm | K15M | | | 36 | 48 | 61 | 74 | 88 | 99 | 100 | 100 |
| F15-50 rpm | Hypromellose | 21.5% | 30.0% | 30 | 36 | 44 | 55 | 69 | 86 | 95 | 100 |
| F15-100 rpm | K100M | | | 33 | 40 | 46 | 57 | 70 | 86 | 95 | 100 |
| F16-50 rpm | Hypromellose | 21.5% | 30.0% | 32 | 37 | 44 | 54 | 67 | 84 | 92 | 97 |
| F16-100 rpm | K200M | | | 35 | 40 | 46 | 56 | 68 | 85 | 93 | 98 |
| F17-50 rpm | Polyethylene | 21.5% | 30.0% | 44 | 55 | 75 | 92 | 97 | 99 | 99 | 99 |
| F17-100 rpm | oxide | | | 48 | 63 | 88 | 99 | 100 | 100 | 100 | 100 |
| F18-50 rpm | Tribehenin | 21.5% | 30.0% | 40 | 45 | 53 | 67 | 83 | 98 | 100 | 100 |
| F18-100 rpm | | | | 50 | 54 | 60 | 77 | 96 | 100 | 100 | 100 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

Result analysis: The viscosity of different hydrophilic skeleton materials of tablets F12, F13, F14, F15, F16, F17 and F18 prepared in Example 2 was investigated in the same proportion (Weight of extended release material accounted for 21.5% to the total weight of table core, Weight of extended release material accounted for 30.0% to the total weight of extended release layer), and the results showed that only when the hydroxypropyl cellulose was a polymer with a viscosity of 13,500-280,000 mPa·s in 2% aqueous solution (20° C.±0.1° C.), the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. The species screening of hydrophilic gel skeleton materials is not obvious.

### Example 3: Preparation of 100 mg Brivaracetam Immediate Release Extended Release Bilayer Tablets

| | | | | | | | |
|---|---|---|---|---|---|---|---|
| Ingredient | F19/mg | F20/mg | F21/mg | F22/mg | F23/mg | F24/mg | F25/mg |

Immediate release layer:

| | | | | | | | |
|---|---|---|---|---|---|---|---|
| Brivaracetam | 25.00 | 25.00 | 25.00 | 25.00 | 25.00 | 25.00 | 25.00 |
| Lactose monohydrate | 51.26 | 51.26 | 51.26 | 51.26 | 51.26 | 51.26 | 51.26 |
| Croscarmellose Sodium | 4.05 | 4.05 | 4.05 | 4.05 | 4.05 | 4.05 | 4.05 |
| Magnesium stearate | 0.68 | 0.68 | 0.68 | 0.68 | 0.68 | 0.68 | 0.68 |
| Anhydrous lactose | 49.28 | 49.28 | 49.28 | 49.28 | 49.28 | 49.28 | 49.28 |
| (additional) | | | | | | | |
| Croscarmellose Sodium | 4.05 | 4.05 | 4.05 | 4.05 | 4.05 | 4.05 | 4.05 |
| (additional) | | | | | | | |
| Magnesium stearate | 0.68 | 0.68 | 0.68 | 0.68 | 0.68 | 0.68 | 0.68 |
| (additional) | | | | | | | |

Extended release layer:

| Brivaracetam | | 75.00 | 75.00 | 75.00 | 75.00 | 75.00 | 75.00 | 75.00 |
|---|---|---|---|---|---|---|---|---|
| Extended | Hypromellose | 188.80 | / | / | / | / | / | / |
| release | K100LV | | | | | | | |
| skeleton | Hypromellose | / | 188.80 | / | / | / | / | / |
| material | K4M | | | | | | | |
| | Hypromellose | / | / | 188.80 | / | / | / | / |
| | K15M | | | | | | | |
| | Hypromellose | / | / | / | 188.80 | / | / | / |
| | K100M | | | | | | | |
| | Hypromellose | / | / | / | / | 188.80 | / | / |
| | K200M | | | | | | | |
| | Polyethylene | / | / | / | / | / | 188.80 | / |
| | oxide | | | | | | | |
| | Tribehenin | / | / | / | / | / | / | 188.80 |

| | | | | | | | |
|---|---|---|---|---|---|---|---|
| Magnesium stearate | 0.79 | 0.79 | 0.79 | 0.79 | 0.79 | 0.79 | 0.79 |
| Silicified microcrystalline | 74.62 | 74.62 | 74.62 | 74.62 | 74.62 | 74.62 | 74.62 |
| cellulose (additional) | 0.79 | 0.79 | 0.79 | 0.79 | 0.79 | 0.79 | 0.79 |
| Magnesium stearate | | | | | | | |
| (additional) | | | | | | | |

Preparation method:
1. Extended release layer:
   1) Brivaracetam through a 20-mesh sieve was mixed with the extended release skeleton material for 30 min, and the magnesium stearate was added to the mixing bucket for 5 min, and the mixture was then dried granulation;
   2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer:
   1) Brivaracetam through a 20-mesh sieve was mixed with the lactose monohydrate and croscarmellose sodium for 30 min, and the magnesium stearate was added to the mixing bucket for 5 min, and the mixture was then dried granulation;
   2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the particles were pressed into a bilayer tablet;
4. Coating: Opadry 85F coating solution was configured to coat the above-mentioned immediate release extended release bilayer tablet core, and the percentage of coating weight gain in the tablet core was 2%

Dissolution detection: According to USP<711> the volume of the medium was 900±9ml, the temperature of the medium was 37.0±0.5° C., and the rotational speed of the paddle method was 50 rpm or 100 rpm. The dissolution characteristics of tablets F19, F20, F21, F22, F23, F24 and F25 in vitro pH 6.8 dissolution medium were determined at 16 h intervals, the results are shown in Table 6.

TABLE 6
Dissolution (%) (n = 3) of example 3 and reference listed drug in pH 6.8 medium

| | | Weight of | | | | | | | | | |
|---|---|---|---|---|---|---|---|---|---|---|---|
| | | extended | Weight of | | | | | | | | |
| | | release | extended | | | | | | | | |
| | | material | release | | | | | | | | |
| | | to the | material to | | | | | | | | |
| | Types of | total | the total | | | | | | | | |
| Group - | extended | weight | weight of | | | | | | | | |
| Dissolution | release | of table | extended | | | | | | | | |
| speed | material | core | release layer | 15 min | 30 min | 1 h | 2 h | 4 h | 8 h | 12 h | 16 h |
| F19-50 rpm | Hypromellose | 39.7% | 55.5% | 36 | 45 | 57 | 74 | 93 | 99 | 99 | 99 |
| F19-100 rpm | K100LV | | | 44 | 52 | 64 | 82 | 96 | 100 | 100 | 100 |
| F20-50 rpm | Hypromellose | 39.7% | 55.5% | 33 | 42 | 52 | 69 | 86 | 96 | 98 | 98 |
| F20-100 rpm | K4M | | | 38 | 47 | 59 | 76 | 90 | 98 | 98 | 98 |
| F21-50 rpm | Hypromellose | 39.7% | 55.5% | 32 | 36 | 43 | 53 | 65 | 81 | 89 | 95 |
| F21-100 rpm | K15M | | | 34 | 39 | 46 | 58 | 69 | 85 | 91 | 95 |
| F22-50 rpm | Hypromellose | 39.7% | 55.5% | 30 | 34 | 40 | 51 | 62 | 77 | 89 | 94 |
| F22-100 rpm | K100M | | | 31 | 35 | 41 | 53 | 63 | 78 | 91 | 96 |
| F23-50 rpm | Hypromellose | 39.7% | 55.5% | 28 | 32 | 39 | 50 | 64 | 78 | 91 | 96 |
| F23-100 rpm | K200M | | | 30 | 32 | 40 | 51 | 66 | 80 | 93 | 98 |
| F24-50 rpm | Polyethylene | 39.7% | 55.5% | 38 | 46 | 58 | 78 | 95 | 96 | 96 | 96 |
| F24-100 rpm | oxide PEO | | | 43 | 51 | 65 | 85 | 99 | 100 | 100 | 100 |
| F25-50 rpm | Tribehenin | 39.7% | 55.5% | 35 | 41 | 47 | 58 | 70 | 86 | 94 | 99 |
| F25-100 rpm | | | | 36 | 45 | 57 | 74 | 93 | 99 | 99 | 99 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

Result analysis: The viscosity of different hydrophilic skeleton materials of tablets F19, F20, F21, F22, F23, F24 and F25 prepared in Example 3 were investigated in the same proportion (Weight of extended release material accounted for 39.7% to the total weight of table core, Weight of extended release material accounted for 55.5% to the total weight of extended release layer), and the results showed that only when the hydroxypropyl cellulose was a polymer with a viscosity of 13,500-280,000 mPa·s in 2% aqueous solution (20° C.±0.1° C.), the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. The species screening of hydrophilic gel skeleton materials is not obvious.

### Example 4: Preparation of 100 mg Brivaracetam Immediate Release Extended Release Bilayer Tablets (K15M, Different Prescription Ratios)

| | | | | | | |
|---|---|---|---|---|---|---|
| Ingredient | F14/mg | F21/mg | F26/mg | F27/mg | F288/mg | F29/mg |

Immediate release layer:

| | | | | | | |
|---|---|---|---|---|---|---|
| Brivaracetam | 25.00 | 25.00 | 25.00 | 25.00 | 25.00 | 25.00 |
| Lactose monohydrate | 51.26 | 51.26 | 51.26 | 51.26 | 51.26 | 51.26 |
| Croscarmellose Sodium | 4.05 | 4.05 | 4.05 | 4.05 | 4.05 | 4.05 |
| Magnesium stearate | 0.68 | 0.68 | 0.68 | 0.68 | 0.68 | 0.68 |
| Anhydrous lactose | 49.28 | 49.28 | 49.28 | 49.28 | 49.28 | 49.28 |
| (additional) | | | | | | |
| Croscarmellose Sodium | 4.05 | 4.05 | 4.05 | 4.05 | 4.05 | 4.05 |
| (additional) | | | | | | |
| Magnesium stearate | 0.68 | 0.68 | 0.68 | 0.68 | 0.68 | 0.68 |
| (additional) | | | | | | |

Extended release layer:

| Brivaracetam | | 75.00 | 75.00 | 75.00 | 75.00 | 75.00 | 75.00 |
|---|---|---|---|---|---|---|---|
| Extended | Hypromellose | 102.00 | 188.80 | 119.65 | 159.39 | 253.23 | 263.42 |
| release | K15M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.79 |
| Silicified microcrystalline | 161.42 | 74.62 | 143.77 | 104.03 | 10.19 | / |
| cellulose (additional) | | | | | | |
| Magnesium stearate | 0.79 | 0.79 | 0.79 | 0.79 | 0.79 | 0.79 |
| (additional) | | | | | | |

Preparation method:
1. Extended release layer:
   1) Brivaracetam through a 20-mesh sieve was mixed with the Hypromellose K15M (trade name: Benecel™ K15M Pharm) for 30 min, and the magnesium stearate was added to the mixing bucket for 5 min, and the mixture was then dried granulation;
   2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer:
   1) Brivaracetam through a 20-mesh sieve was mixed with the lactose monohydrate and croscarmellose sodium for 30 min, and the magnesium stearate was added to the mixing bucket for 5 min, and the mixture was then dried granulation;
   2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711> the volume of the medium was 900±9 ml, the temperature of the medium was 37.0±0.5° C., and the rotational speed of the paddle method was 50 rpm or 100 rpm. The dissolution characteristics of tablets F14, F21, F26, F27, F28 and F29 in vitro pH 6.8 dissolution medium were determined at 16 h intervals.

TABLE 7
Dissolution (%) (n = 3) of example 4 and reference listed drug in pH 6.8 medium

| | | Weight of | Weight of | | | | | | | | |
|---|---|---|---|---|---|---|---|---|---|---|---|
| | | extended | extended | | | | | | | | |
| | | release | release | | | | | | | | |
| | Types of | material to | material to the | | | | | | | | |
| | extended | the total | total weight of | | | | | | | | |
| | release | weight of | extended | | | | | | | | |
| Group | material | table core | release layer | 15 min | 30 min | 1 h | 2 h | 4 h | 8 h | 12 h | 16 h |
| F14-50 rpm | K15M | 21.5% | 30.0% | 33 | 45 | 56 | 68 | 78 | 94 | 99 | 99 |
| F14-100 rpm | | | | 36 | 48 | 61 | 74 | 88 | 99 | 100 | 100 |
| F21-50 rpm | K15M | 39.7% | 55.5% | 32 | 36 | 43 | 53 | 65 | 81 | 89 | 95 |
| F21-100 rpm | | | | 34 | 39 | 46 | 58 | 69 | 85 | 91 | 95 |
| F26-50 rpm | K15M | 25.2% | 35.2% | 32 | 41 | 51 | 63 | 74 | 87 | 95 | 98 |
| F26-100 rpm | | | | 35 | 44 | 53 | 67 | 79 | 90 | 97 | 99 |
| F27-50 rpm | K15M | 33.6% | 46.9% | 33 | 38 | 45 | 56 | 67 | 84 | 94 | 98 |
| F27-100 rpm | | | | 35 | 40 | 47 | 60 | 72 | 86 | 96 | 99 |
| F28-50 rpm | K15M | 53.3% | 74.5% | 32 | 36 | 42 | 51 | 62 | 77 | 86 | 93 |
| F28-100 rpm | | | | 33 | 37 | 43 | 52 | 63 | 78 | 87 | 95 |
| F29-50 rpm | K15M | 55.5% | 77.5% | 31 | 35 | 41 | 50 | 60 | 74 | 85 | 91 |
| F29-100 rpm | | | | 32 | 37 | 41 | 51 | 62 | 75 | 86 | 93 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F14, F21, F26, F27, F28 and F29 prepared in example 4 show that brivaracetam extended release tablets were prepared by hydrophilic gel polymer hypromellose, in which the amount of hypromellose polymer is 55.5%-77.5%/weight relative to the total weight of the extended release layer, or the amount of hypromellose polymer is 39.7%-55.5%/weight relative to the total weight of the tablet core, and the hypromellose polymer used is usually in 2% aqueous solution (20° C.±0.1° C.) with a viscosity of 13,500-280,000 mPa·s, the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. The proportion screening of hydrophilic gel skeleton materials is not obvious

### Example 5: Preparation of 100 mg Brivaracetam Immediate Release Extended Release Bilayer Tablets (K100M, Different Prescription Ratios)

| | | | | | | |
|---|---|---|---|---|---|---|
| Ingredient | F15/mg | F22/mg | F30/mg | F31/mg | F32/mg | F33/mg |

Immediate release layer:

| | | | | | | |
|---|---|---|---|---|---|---|
| Brivaracetam | 25.00 | 25.00 | 25.00 | 25.00 | 25.00 | 25.00 |
| Lactose monohydrate | 51.26 | 51.26 | 51.26 | 51.26 | 51.26 | 51.26 |
| Croscarmellose Sodium | 4.05 | 4.05 | 4.05 | 4.05 | 4.05 | 4.05 |
| Magnesium stearate | 0.68 | 0.68 | 0.68 | 0.68 | 0.68 | 0.68 |
| Anhydrous lactose | 49.28 | 49.28 | 49.28 | 49.28 | 49.28 | 49.28 |
| (additional) | | | | | | |
| Croscarmellose Sodium | 4.05 | 4.05 | 4.05 | 4.05 | 4.05 | 4.05 |
| (additional) | | | | | | |
| Magnesium stearate | 0.68 | 0.68 | 0.68 | 0.68 | 0.68 | 0.68 |
| (additional) | | | | | | |

Extended release layer:

| Brivaracetam | | 75.00 | 75.00 | 75.00 | 75.00 | 75.00 | 75.00 |
|---|---|---|---|---|---|---|---|
| Extended | Hypromellose | 102.00 | 188.80 | 119.65 | 159.39 | 253.23 | 263.42 |
| release | K100M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.79 |
| Silicified microcrystalline | 161.42 | 74.62 | 143.77 | 104.03 | 10.19 | / |
| cellulose (additional) | | | | | | |
| Magnesium stearate | 0.79 | 0.79 | 0.79 | 0.79 | 0.79 | 0.79 |
| (additional) | | | | | | |

Preparation method:
1. Extended release layer: 1) TBrivaracetam through a 20-mesh sieve was mixed with the Hypromellose K100M (trade name: Benecel™MK100M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing barrel and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711> the volume of the medium was 900±9 ml, the temperature of the medium was 37.0±0.5° C., and the rotational speed of the paddle method was 50 rpm or 100 rpm. The dissolution characteristics of tablets F15, F22, F30, F31, F32 and F33 in vitro pH 6.8 dissolution medium were determined at 16 h intervals.

TABLE 8
Dissolution (%) (n = 3) of example 5 and reference listed drug in pH 6.8 medium

| | | Weight of | Weight of | | | | | | | | |
|---|---|---|---|---|---|---|---|---|---|---|---|
| | | extended | extended | | | | | | | | |
| | | release | release material | | | | | | | | |
| | Types of | material to | to the total | | | | | | | | |
| | extended | the total | weight of | | | | | | | | |
| | release | weight of | extended | | | | | | | | |
| Group | material | table core | release layer | 15 min | 30 min | 1 h | 2 h | 4 h | 8 h | 12 h | 16 h |
| F15-50 rpm | K100M | 21.5% | 30.0% | 30 | 36 | 44 | 55 | 69 | 86 | 95 | 100 |
| F15-100 rpm | | | | 33 | 40 | 46 | 57 | 70 | 86 | 95 | 100 |
| F22-50 rpm | K100M | 39.7% | 55.5% | 30 | 34 | 40 | 51 | 62 | 77 | 89 | 94 |
| F22-100 rpm | | | | 31 | 35 | 41 | 53 | 63 | 78 | 91 | 96 |
| F30-50 rpm | K100M | 25.2% | 35.2% | 29 | 35 | 42 | 53 | 66 | 83 | 93 | 98 |
| F30-100 rpm | | | | 33 | 38 | 46 | 55 | 67 | 86 | 95 | 100 |
| F31-50 rpm | K100M | 33.6% | 46.9% | 30 | 34 | 41 | 53 | 64 | 79 | 90 | 95 |
| F31-100 rpm | | | | 30 | 35 | 42 | 55 | 69 | 81 | 92 | 100 |
| F32-50 rpm | K100M | 53.3% | 74.5% | 27 | 30 | 36 | 49 | 58 | 75 | 86 | 93 |
| F32-100 rpm | | | | 28 | 30 | 37 | 50 | 59 | 76 | 88 | 95 |
| F33-50 rpm | K100M | 55.5% | 77.5% | 26 | 29 | 34 | 47 | 57 | 73 | 85 | 90 |
| F33-100 rpm | | | | 26 | 30 | 35 | 48 | 59 | 74 | 86 | 93 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F15, F22, F30, F31, F32 and F33 prepared in example 5 show that brivaracetam extended release tablets were prepared by hydrophilic gel polymer hypromellose, in which the amount of hypromellose polymer is 46.9%-77.5%/weight relative to the total weight of the extended release layer, or the amount of hypromellose polymer is 33.6%-55.5%/weight relative to the total weight of the tablet core, and the hypromellose polymer used is usually in 2% aqueous solution (20° C.±0.1° C.) with a viscosity of 13,500-280,000 mPa·s, the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. The proportion screening of hydrophilic gel skeleton materials is not obvious

### Example 6: Preparation of 100 mg Brivaracetam Immediate Release Extended Release Bilayer Tablets (K200M, Different Prescription Ratios)

| | | | | | | |
|---|---|---|---|---|---|---|
| Ingredient | F16/mg | F23/mg | F34/mg | F35/mg | F36/mg | F37/mg |

Immediate release layer:

| | | | | | | |
|---|---|---|---|---|---|---|
| Brivaracetam | 25.00 | 25.00 | 25.00 | 25.00 | 25.00 | 25.00 |
| Lactose monohydrate | 51.26 | 51.26 | 51.26 | 51.26 | 51.26 | 11.26 |
| Croscarmellose Sodium | 4.05 | 4.05 | 4.05 | 4.05 | 4.05 | 4.05 |
| Magnesium stearate | 0.68 | 0.68 | 0.68 | 0.68 | 0.68 | 0.68 |
| Anhydrous lactose | 49.28 | 49.28 | 132.43 | 92.43 | 13.53 | 13.53 |
| (additional) | | | | | | |
| Croscarmellose Sodium | 4.05 | 4.05 | 4.05 | 4.05 | 4.05 | 4.05 |
| (additional) | | | | | | |
| Magnesium stearate | 0.68 | 0.68 | 0.68 | 0.68 | 0.68 | 0.68 |
| (additional) | | | | | | |

Extended release layer:

| Brivaracetam | | 75.00 | 75.00 | 75.00 | 75.00 | 75.00 | 75.00 |
|---|---|---|---|---|---|---|---|
| Extended | Hypromellose | 102.00 | 188.80 | 119.65 | 159.39 | 253.23 | 263.42 |
| release | K200M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.79 |
| Silicified microcrystalline | 161.42 | 74.62 | 143.77 | 104.03 | 10.19 | / |
| cellulose (additional) | | | | | | |
| Magnesium stearate | 0.79 | 0.79 | 0.79 | 0.79 | 0.79 | 0.79 |

(additional)

Preparation method: 1. Extended release layer: 1) Brivaracetam through a 20-mesh sieve was mixed with the hypromellose K200M (trade name: Benecel™ K200M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711>, the volume of the medium was 900±9 ml, the temperature of the medium was 37.0±0.5° C., and the rotational speed of the paddle method was 50 rpm or 100 rpm. The dissolution characteristics of tablets F16, F23, F34, F35, F36 and F37 in vitro pH 6.8 dissolution medium were determined at 16 h intervals.

TABLE 9
Dissolution (%) (n = 3) of example 6 and reference listed drug in pH 6.8 medium

| | | | Weight of | | | | | | | | |
|---|---|---|---|---|---|---|---|---|---|---|---|
| | | Weight of | extended | | | | | | | | |
| | | extended | release | | | | | | | | |
| | | release | material to | | | | | | | | |
| | Types of | material to | the total | | | | | | | | |
| Group - | extended | the total | weight of | | | | | | | | |
| Dissolution | release | weight of | extended | | | | | | | | |
| speed | material | table core | release layer | 15 min | 30 min | 1 h | 2 h | 4 h | 8 h | 12 h | 16 h |
| F16-50 rpm | K200M | 21.5% | 30.0% | 32 | 37 | 44 | 54 | 67 | 84 | 92 | 97 |
| F16-100 rpm | | | | 35 | 40 | 46 | 56 | 68 | 85 | 93 | 98 |
| F23-50 rpm | K200M | 39.7% | 55.5% | 28 | 32 | 39 | 50 | 64 | 78 | 91 | 96 |
| F23-100 rpm | | | | 30 | 32 | 40 | 51 | 66 | 80 | 93 | 98 |
| F34-50 rpm | K200M | 25.2% | 35.2% | 31 | 36 | 42 | 52 | 65 | 82 | 91 | 96 |
| F34-100 rpm | | | | 33 | 38 | 45 | 55 | 67 | 84 | 93 | 98 |
| F35-50 rpm | K200M | 33.6% | 46.9% | 30 | 33 | 41 | 51 | 65 | 80 | 92 | 97 |
| F35-100 rpm | | | | 30 | 34 | 43 | 52 | 66 | 82 | 92 | 98 |
| F36-50 rpm | K200M | 53.3% | 74.5% | 26 | 29 | 35 | 49 | 57 | 75 | 85 | 93 |
| F36-100 rpm | | | | 27 | 29 | 35 | 50 | 58 | 75 | 87 | 95 |
| F37-50 rpm | K200M | 55.5% | 77.5% | 25 | 29 | 34 | 47 | 56 | 75 | 86 | 91 |
| F37-100 rpm | | | | 25 | 30 | 36 | 47 | 57 | 75 | 87 | 93 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F16, F23, F34, F35, F36 and F37 prepared in example 6 show that brivaracetam extended release tablets were prepared by hydrophilic gel polymer hypromellose, in which the amount of hypromellose polymer is 30.0%-77.5%/weight relative to the total weight of the extended release layer, or the amount of hypromellose polymer is 21.5%-55.5%/weight relative to the total weight of the tablet core, and the hypromellose polymer used is usually in 2% aqueous solution (20° C.±0.1° C.) with a viscosity of 13,500-280,000 mPa·s, the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. The proportion screening of hydrophilic gel skeleton materials is not obvious

### Example 7: Preparation of 100 mg Brivaracetam Immediate Release Extended Release Bilayer Tablets (K15M, Different ER and IR Ratios)

| | | | | | | |
|---|---|---|---|---|---|---|
| Ingredient | F21/mg | F38/mg | F39/mg | F40/mg | F41/mg | F42/mg |

Immediate release layer:

| | | | | | | |
|---|---|---|---|---|---|---|
| Brivaracetam | 25.00 | 50.00 | 33.00 | 20.00 | 16.00 | / |
| Lactose monohydrate | 51.26 | 51.26 | 51.26 | 51.26 | 51.26 | / |
| Croscarmellose Sodium | 4.05 | 4.05 | 4.05 | 4.05 | 4.05 | / |
| Magnesium stearate | 0.68 | 0.68 | 0.68 | 0.68 | 0.68 | / |
| Anhydrous lactose | 49.28 | 49.28 | 49.28 | 49.28 | 49.28 | / |
| (additional) | | | | | | |
| Croscarmellose Sodium | 4.05 | 4.05 | 4.05 | 4.05 | 4.05 | / |
| (additional) | | | | | | |
| Magnesium stearate | 0.68 | 0.68 | 0.68 | 0.68 | 0.68 | / |
| (additional) | | | | | | |

Extended release layer:

| Brivaracetam | | 75.00 | 50.00 | 67.00 | 80.00 | 84.00 | 100.00 |
|---|---|---|---|---|---|---|---|
| Extended | Hypromellose | 188.80 | 188.80 | 188.80 | 188.80 | 188.80 | 224.76 |
| release | K15M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.94 |
| Silicified microcrystalline | 74.62 | 99.62 | 82.62 | 69.62 | 65.62 | 78.36 |
| cellulose (additional) | | | | | | |
| Magnesium stearate | 0.79 | 0.79 | 0.79 | 0.79 | 0.79 | 0.94 |
| (additional) | | | | | | |

Preparation method: 1. Extended release layer: 1) Brivaracetam through a 20-mesh sieve was mixed with the hypromellose K15M (trade name: Benecel™ K15M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711> the volume of the medium was 900±9 ml, the temperature of the medium was 37.0±0.5° C., and the rotational speed of the paddle method was 50 rpm or 100 rpm. The dissolution characteristics of tablets F21, F38, F39, F40, F41 and F42 in vitro pH 6.8 dissolution medium were determined at 16 h intervals.

TABLE 10
Dissolution (%) (n = 3) of example 7 and reference listed drug in pH 6.8 medium

| | IR | ER | | | | | | | | |
|---|---|---|---|---|---|---|---|---|---|---|
| Group - | specification | specification | | | | | | | | |
| Dissolution | to the total | to the total | | | | | | | | |
| speed | specification | specification | 15 min | 30 min | 1 h | 2 h | 4 h | 8 h | 12 h | 16 h |
| F21-50 rpm | 25.0% | 75.0% | 32 | 36 | 43 | 53 | 65 | 81 | 89 | 95 |
| F21-100 rpm | | | 34 | 39 | 46 | 58 | 69 | 85 | 91 | 95 |
| F38-50 rpm | 50.0% | 50.0% | 55 | 60 | 64 | 70 | 79 | 89 | 96 | 99 |
| F38-100 rpm | | | 58 | 61 | 67 | 76 | 83 | 94 | 97 | 100 |
| F39-50 rpm | 33.0% | 67.0% | 39 | 40 | 47 | 56 | 68 | 83 | 92 | 98 |
| F39-100 rpm | | | 41 | 43 | 49 | 58 | 71 | 86 | 96 | 99 |
| F40-50 rpm | 20.0% | 80.0% | 27 | 31 | 39 | 49 | 61 | 79 | 88 | 95 |
| F40-100 rpm | | | 30 | 34 | 42 | 51 | 63 | 80 | 89 | 96 |
| F41-50 rpm | 16.0% | 84.0% | 25 | 29 | 37 | 48 | 58 | 76 | 91 | 96 |
| F41-100 rpm | | | 28 | 32 | 40 | 52 | 62 | 80 | 92 | 97 |
| F42-50 rpm | 0.0% | 100.0% | 10 | 16 | 24 | 36 | 55 | 76 | 84 | 92 |
| F42-100 rpm | | | 13 | 20 | 28 | 39 | 59 | 79 | 88 | 95 |
| Reference | / | / | 101 | — | — | — | — | — | — | — |
| listed drug | | | | | | | | | | |
| (trade name: | | | | | | | | | | |
| BRIVIACT) | | | | | | | | | | |

The results of the tablets F21, F38, F39, F40, F41 and F42 prepared in example 7 show that when the IR ratio is 16.0%-25.0%

$$( IR ratio = \frac{IR specification}{IR specification + ER specification} \times 100 \% ) ,$$

the brivaracetam immediate release extended release bilayer tablet prepared by the present invention can achieve the effect of rapid onset and long-term extended release. And the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient.

### Example 8: Preparation of 100 mg Brivaracetam Immediate Release Extended Release Bilayer Tablets (K100M, Different ER and IR Ratios)

| | | | | | | |
|---|---|---|---|---|---|---|
| Ingredient | F22/mg | F43/mg | F44/mg | F45/mg | F46/mg | F47/mg |

Immediate release layer:

| | | | | | | |
|---|---|---|---|---|---|---|
| Brivaracetam | 25.00 | 50.00 | 33.00 | 20.00 | 16.00 | / |
| Lactose monohydrate | 51.26 | 51.26 | 51.26 | 51.26 | 51.26 | / |
| Croscarmellose Sodium | 4.05 | 4.05 | 4.05 | 4.05 | 4.05 | / |
| Magnesium stearate | 0.68 | 0.68 | 0.68 | 0.68 | 0.68 | / |
| Anhydrous lactose | 49.28 | 49.28 | 49.28 | 49.28 | 49.28 | / |
| (additional) | | | | | | |
| Croscarmellose Sodium | 4.05 | 4.05 | 4.05 | 4.05 | 4.05 | / |
| (additional) | | | | | | |
| Magnesium stearate | 0.68 | 0.68 | 0.68 | 0.68 | 0.68 | / |
| (additional) | | | | | | |

Extended release layer:

| Brivaracetam | | 75.00 | 50.00 | 67.00 | 80.00 | 84.00 | 100.00 |
|---|---|---|---|---|---|---|---|
| Extended | Hypromellose | 188.80 | 188.80 | 188.80 | 188.80 | 188.80 | 224.76 |
| release | K100M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.94 |
| Silicified microcrystalline | 74.62 | 99.62 | 82.62 | 69.62 | 65.62 | 78.36 |
| cellulose (additional) | | | | | | |
| Magnesium stearate | 0.79 | 0.79 | 0.79 | 0.79 | 0.79 | 0.94 |
| (additional) | | | | | | |

Preparation method: 1. Extended release layer: 1) Brivaracetam through a 20-mesh sieve was mixed with the hypromellose K100M (trade name: Benecel™ K100M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711>, the volume of the medium was 900±9 ml, the temperature of the medium was 37.0±0.5° C., and the rotational speed of the paddle method was 50 rpm or 100 rpm. The dissolution characteristics of tablets F22, F43, F44, F45, F46 and F47 in vitro pH 6.8 dissolution medium were determined at 16 h intervals.

TABLE 11
Dissolution (%) (n = 8) of example 8 and reference listed drug in pH 6.8 medium

| | IR | ER | | | | | | | | |
|---|---|---|---|---|---|---|---|---|---|---|
| Group - | specification | specification | | | | | | | | |
| Dissolution | to the total | to the total | | | | | | | | |
| speed | specification | specification | 15 min | 30 min | 1 h | 2 h | 4 h | 8 h | 12 h | 16 h |
| F22-50 rpm | 25.0% | 75.0% | 30 | 34 | 40 | 51 | 62 | 77 | 89 | 94 |
| F22-100 rpm | | | 31 | 35 | 41 | 53 | 63 | 78 | 91 | 96 |
| F43-50 rpm | 50.0% | 50.0% | 53 | 58 | 60 | 68 | 77 | 88 | 95 | 99 |
| F43-100 rpm | | | 55 | 60 | 62 | 72 | 80 | 91 | 96 | 99 |
| F44-50 rpm | 33.0% | 67.0% | 36 | 41 | 45 | 57 | 69 | 84 | 92 | 96 |
| F44-100 rpm | | | 38 | 42 | 46 | 57 | 70 | 85 | 94 | 98 |
| F45-50 rpm | 20.0% | 80.0% | 26 | 31 | 39 | 48 | 59 | 78 | 89 | 95 |
| F45-100 rpm | | | 28 | 33 | 40 | 49 | 61 | 80 | 91 | 97 |
| F46-50 rpm | 16.0% | 84.0% | 23 | 28 | 36 | 46 | 57 | 75 | 90 | 95 |
| F46-100 rpm | | | 27 | 30 | 37 | 46 | 58 | 76 | 92 | 98 |
| F47-50 rpm | 0.0% | 100.0% | 8 | 14 | 22 | 33 | 51 | 70 | 81 | 89 |
| F47-100 rpm | | | 10 | 16 | 25 | 36 | 53 | 72 | 83 | 93 |
| Reference | / | / | 101 | — | — | — | — | — | — | — |
| listed drug | | | | | | | | | | |
| (trade name: | | | | | | | | | | |
| BRIVIACT) | | | | | | | | | | |

The results of the tablets F22, F43, F44, F45, F46 and F47 prepared in example 8 show that when the IR ratio is 16.0%-33.0%

$$( IR ratio = \frac{IR specification}{IR specification + ER specification} \times 100 \% ) ,$$

the brivaracetam immediate release extended release bilayer tablet prepared by the present invention can achieve the effect of rapid onset and long-term extended release. And the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient.

### Example 9: Preparation of 100 mg Brivaracetam Immediate Release Extended Release Bilayer Tablets (K200M, Different ER and IR Ratios)

| | | | | | | |
|---|---|---|---|---|---|---|
| Ingredient | F23/mg | F48/mg | F49/mg | F50/mg | F51/mg | F52/mg |

Immediate release layer:

| | | | | | | |
|---|---|---|---|---|---|---|
| Brivaracetam | 25.00 | 50.00 | 33.00 | 20.00 | 16.00 | / |
| Lactose monohydrate | 51.26 | 51.26 | 51.26 | 51.26 | 51.26 | / |
| Croscarmellose Sodium | 4.05 | 4.05 | 4.05 | 4.05 | 4.05 | / |
| Magnesium stearate | 0.68 | 0.68 | 0.68 | 0.68 | 0.68 | / |
| Anhydrous lactose | 49.28 | 49.28 | 49.28 | 49.28 | 49.28 | / |
| (additional) | | | | | | |
| Croscarmellose Sodium | 4.05 | 4.05 | 4.05 | 4.05 | 4.05 | / |
| (additional) | | | | | | |
| Magnesium stearate | 0.68 | 0.68 | 0.68 | 0.68 | 0.68 | / |
| (additional) | | | | | | |

Extended release layer:

| Brivaracetam | | 75.00 | 50.00 | 67.00 | 80.00 | 84.00 | 100.00 |
|---|---|---|---|---|---|---|---|
| Extended | Hypromellose | 188.80 | 188.80 | 188.80 | 188.80 | 188.80 | 224.76 |
| release | K200M | | | | | | |
| skeleton | | | | | | | |
| material | | | | | | | |

| | | | | | | |
|---|---|---|---|---|---|---|
| Magnesium stearate | 0.79 | 0.79 | 0.79 | 0.79 | 0.79 | 0.94 |
| Silicified microcrystalline | 74.62 | 99.62 | 82.62 | 69.62 | 65.62 | 78.36 |
| cellulose (additional) | | | | | | |
| Magnesium stearate | 0.79 | 0.79 | 0.79 | 0.79 | 0.79 | 0.94 |
| (additional) | | | | | | |

Preparation method: 1. Extended release layer: 1) Brivaracetam through a 20-mesh sieve was mixed with the hypromellose K200M (trade name: Benecel™ K200M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711> the volume of the medium was 900±9 ml, the temperature of the medium was 37.0±0.5° C., and the rotational speed of the paddle method was 50 rpm or 100 rpm. The dissolution characteristics of tablets F23, F48, F49, F50, F51 and F52 in vitro pH 6.8 dissolution medium were determined at 16 h intervals.

TABLE 12
Dissolution (%) (n = 3) of example 9 and reference listed drug in pH 6.8 medium

| | IR | ER | | | | | | | | |
|---|---|---|---|---|---|---|---|---|---|---|
| | specification | specification | | | | | | | | |
| | to the total | to the total | | | | | | | | |
| Group | specification | specification | 15 min | 30 min | 1 h | 2 h | 4 h | 8 h | 12 h | 16 h |
| F23-50 rpm | 25.0% | 75.0% | 28 | 32 | 39 | 50 | 64 | 78 | 91 | 96 |
| F23-100 rpm | | | 30 | 32 | 40 | 51 | 66 | 80 | 93 | 98 |
| F48-50 rpm | 50.0% | 50.0% | 53 | 57 | 58 | 67 | 76 | 88 | 95 | 99 |
| F48-100 rpm | | | 54 | 59 | 60 | 70 | 78 | 90 | 96 | 99 |
| F49-50 rpm | 33.0% | 67.0% | 36 | 40 | 45 | 56 | 67 | 83 | 93 | 96 |
| F49-100 rpm | | | 37 | 41 | 45 | 56 | 69 | 84 | 93 | 97 |
| F50-50 rpm | 20.0% | 80.0% | 26 | 30 | 38 | 46 | 58 | 76 | 88 | 95 |
| F50-100 rpm | | | 27 | 31 | 39 | 48 | 59 | 78 | 90 | 96 |
| F51-50 rpm | 16.0% | 84.0% | 23 | 28 | 35 | 46 | 56 | 75 | 89 | 95 |
| F51-100 rpm | | | 25 | 28 | 36 | 47 | 58 | 77 | 91 | 97 |
| F52-50 rpm | 0.0% | 100.0% | 7 | 12 | 21 | 31 | 47 | 67 | 79 | 89 |
| F52-100 rpm | | | 9 | 14 | 23 | 34 | 50 | 69 | 81 | 92 |
| Reference | / | / | 101 | — | — | — | — | — | — | — |
| listed drug | | | | | | | | | | |
| (trade name: | | | | | | | | | | |
| BRIVIACT) | | | | | | | | | | |

The results of the tablets F22, F43, F44, F45, F46 and F47 prepared in example 9 show that when the IR ratio is 16.0%-33.0%

$$( IR ratio = \frac{IR specification}{IR specification + ER specification} \times 100 \% ) ,$$

the brivaracetam immediate release extended release bilayer tablet prepared by the present invention can achieve the effect of rapid onset and long-term extended release. And the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient.

### Example 10: Preparation of 100 mg Brivaracetam Immediate Release Extended Release Bilayer Tablets (K15M, Different Specifications)

| | | | | | |
|---|---|---|---|---|---|
| Ingredient | F21/mg | F53/mg | F54/mg | F55/mg | F56/mg |

Immediate release layer:

| | | | | | |
|---|---|---|---|---|---|
| Brivaracetam | 25.00 | 22.50 | 27.50 | 30.00 | 32.50 |
| Lactose monohydrate | 51.26 | 51.26 | 51.26 | 51.26 | 51.26 |
| Croscarmellose Sodium | 4.05 | 4.05 | 4.05 | 4.05 | 4.05 |
| Magnesium stearate | 0.68 | 0.68 | 0.68 | 0.68 | 0.68 |
| Anhydrous lactose | 49.28 | 51.78 | 46.78 | 44.28 | 41.78 |
| (additional) | | | | | |
| Croscarmellose Sodium | 4.05 | 4.05 | 4.05 | 4.05 | 4.05 |
| (additional) | | | | | |
| Magnesium stearate | 0.68 | 0.68 | 0.68 | 0.68 | 0.68 |
| (additional) | | | | | |

Extended release layer:

| Brivaracetam | | 75.00 | 67.50 | 82.50 | 90.00 | 97.50 |
|---|---|---|---|---|---|---|
| Extended release | Hypromellose | 188.80 | 188.80 | 188.80 | 188.80 | 188.80 |
| skeleton material | K15M | | | | | |

| | | | | | |
|---|---|---|---|---|---|
| Magnesium stearate | 0.79 | 0.79 | 0.79 | 0.79 | 0.79 |
| Silicified microcrystalline | 74.62 | 82.12 | 67.12 | 59.62 | 52.12 |
| cellulose (additional) | | | | | |
| Magnesium stearate | 0.79 | 0.79 | 0.79 | 0.79 | 0.79 |
| (additional) | | | | | |

Preparation method: 1. Extended release layer: 1) Brivaracetam through a 20-mesh sieve was mixed with the hypromellose K15M (trade name: Benecel™ K15M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. The two parts of the pellet were pressed into a bilayer tablet;
4. Opadry 85F coating solution was configured to coat the above-mentioned immediate release extended release bilayer tablet core, and the percentage of coating weight gain in the tablet core was 2%.

According to USP<711>, the volume of the medium was 900±9 ml, the temperature of the medium was 37.0±0.5° C., and the rotational speed of the paddle method was 50 rpm or 100 rpm. The dissolution characteristics of tablets F21, F53, F54, F55 and F56 in vitro pH 6.8 dissolution medium were determined at 16 h intervals.

TABLE 13
Dissolution (%) (n = 3) of example 10 and reference listed drug in pH 6.8 medium

| Group - | IR | ER | General | | | | | | | | |
|---|---|---|---|---|---|---|---|---|---|---|---|
| Dissolution | Specifications/ | Specifications/ | Specifications/ | | | | | | | | |
| speed | mg | mg | mg | 15 min | 30 min | 1 h | 2 h | 4 h | 8 h | 12 h | 16 h |
| F21-50 rpm | 25.0 | 75.0 | 100.0 | 32 | 36 | 43 | 53 | 65 | 81 | 89 | 95 |
| F21-100 rpm | | | | 34 | 39 | 46 | 58 | 69 | 85 | 91 | 95 |
| F53-50 rpm | 22.5 | 67.5 | 90.0 | 32 | 36 | 44 | 54 | 67 | 82 | 91 | 96 |
| F53-100 rpm | | | | 35 | 39 | 46 | 59 | 69 | 85 | 93 | 97 |
| F54-50 rpm | 27.5 | 82.5 | 110.0 | 31 | 35 | 42 | 53 | 63 | 80 | 90 | 94 |
| F54-100 rpm | | | | 33 | 38 | 45 | 57 | 67 | 81 | 92 | 96 |
| F55-50 rpm | 30.0 | 90.0 | 120.0 | 31 | 35 | 42 | 52 | 64 | 80 | 88 | 94 |
| F55-100 rpm | | | | 34 | 40 | 46 | 56 | 66 | 82 | 91 | 95 |
| F56-50 rpm | 32.5 | 97.5 | 130.0 | 30 | 36 | 43 | 51 | 64 | 79 | 89 | 94 |
| F56-100 rpm | | | | 30 | 38 | 47 | 55 | 67 | 83 | 92 | 95 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F21, F53, F54, F55 and F56 prepared in example 10 show that when the specification is 90 mg-120 mg (specification=IR specification+ER specification), the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient.

### Example 11: Preparation of 100 mg Brivaracetam Immediate Release Extended Release Bilayer Tablets (K100M, Different Specifications)

| | | | | | |
|---|---|---|---|---|---|
| Ingredient | F22/mg | F57/mg | F58/mg | F59/mg | F60/mg |

Immediate release layer:

| | | | | | |
|---|---|---|---|---|---|
| Brivaracetam | 25.00 | 22.50 | 27.50 | 30.00 | 32.50 |
| Lactose monohydrate | 51.26 | 51.26 | 51.26 | 51.26 | 51.26 |
| Croscarmellose Sodium | 4.05 | 4.05 | 4.05 | 4.05 | 4.05 |
| Magnesium stearate | 0.68 | 0.68 | 0.68 | 0.68 | 0.68 |
| Anhydrous lactose | 49.28 | 51.78 | 46.78 | 44.28 | 41.78 |
| (additional) | | | | | |
| Croscarmellose Sodium | 4.05 | 4.05 | 4.05 | 4.05 | 4.05 |
| (additional) | | | | | |
| Magnesium stearate | 0.68 | 0.68 | 0.68 | 0.68 | 0.68 |
| (additional) | | | | | |

Extended release layer:

| Brivaracetam | | 75.00 | 67.50 | 82.50 | 90.00 | 97.50 |
|---|---|---|---|---|---|---|
| Extended release | Hypromellose | 188.80 | 188.80 | 188.80 | 188.80 | 188.80 |
| skeleton material | K100M | | | | | |

| | | | | | |
|---|---|---|---|---|---|
| Magnesium stearate | 0.79 | 0.79 | 0.79 | 0.79 | 0.79 |
| Silicified microcrystalline | 74.62 | 82.12 | 67.12 | 59.62 | 52.12 |
| cellulose (additional) | | | | | |
| Magnesium stearate | 0.79 | 0.79 | 0.79 | 0.79 | 0.79 |
| (additional) | | | | | |

Preparation method: 1. Extended release layer: 1) Brivaracetam through a 20-mesh sieve was mixed with the hypromellose K100M (trade name: Benecel™K100M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. Compressing tablet: The two parts of the pellet were pressed into a bilayer tablet;
4. Coating: Opadry 85F coating solution was configured to coat the above-mentioned immediate release extended release bilayer tablet core, and the percentage of coating weight gain in the tablet core was 2%.

According to USP<711>, the volume of the medium was 900±9 ml, the temperature of the medium was 37.0±0.5° C., and the rotational speed of the paddle method was 50 rpm or 100 rpm. The dissolution characteristics of tablets F22, F57, F58, F59 and F60 in vitro pH 6.8 dissolution medium were determined at 16 h intervals.

TABLE 14
Dissolution (%) (n = 3) of example 11 and reference listed drug in pH 6.8 medium

| Group - | IR | ER | General | | | | | | | | |
|---|---|---|---|---|---|---|---|---|---|---|---|
| Dissolution | specifications/ | specifications/ | Specifications/ | | | | | | | | |
| speed | mg | mg | mg | 15 min | 30 min | 1 h | 2 h | 4 h | 8 h | 12 h | 16 h |
| F22-50 rpm | 25.0 | 75.0 | 100.0 | 28 | 32 | 39 | 50 | 64 | 78 | 91 | 96 |
| F22-100 rpm | | | | 30 | 32 | 40 | 51 | 66 | 80 | 93 | 98 |
| F57-50 rpm | 22.5 | 67.5 | 90.0 | 29 | 33 | 41 | 52 | 66 | 79 | 93 | 97 |
| F57-100 rpm | | | | 31 | 34 | 41 | 53 | 67 | 80 | 95 | 99 |
| F58-50 rpm | 27.5 | 82.5 | 110.0 | 28 | 31 | 39 | 49 | 63 | 78 | 90 | 96 |
| F58-100 rpm | | | | 30 | 31 | 40 | 50 | 64 | 78 | 92 | 98 |
| F59-50 rpm | 30.0 | 90.0 | 120.0 | 28 | 30 | 38 | 49 | 63 | 77 | 89 | 96 |
| F59-100 rpm | | | | 29 | 31 | 40 | 50 | 64 | 79 | 92 | 97 |
| F60-50 rpm | 32.5 | 97.5 | 130.0 | 29 | 30 | 39 | 49 | 63 | 77 | 91 | 97 |
| F60-100 rpm | | | | 29 | 29 | 40 | 49 | 63 | 78 | 93 | 96 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F22, F57, F58, F59 and F60 prepared in example 11 show that when the specification is 90 mg-130 mg (specification=IR specification+ER specification), the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. Because the 130 mg specification has a higher exposure compared to RLD, the preferred specification is 90 mg to 120 mg.

### Example 12: Preparation of 100 mg Brivaracetam Immediate Release Extended Release Bilayer Tablets (K200M, Different Specifications)

| | | | | | |
|---|---|---|---|---|---|
| Ingredient | F23/mg | F61/mg | F62/mg | F63/mg | F64/mg |

Immediate release layer:

| | | | | | |
|---|---|---|---|---|---|
| Brivaracetam | 25.00 | 22.50 | 27.50 | 30.00 | 32.50 |
| Lactose monohydrate | 51.26 | 51.26 | 51.26 | 51.26 | 51.26 |
| Croscarmellose Sodium | 4.05 | 4.05 | 4.05 | 4.05 | 4.05 |
| Magnesium stearate | 0.68 | 0.68 | 0.68 | 0.68 | 0.68 |
| Anhydrous lactose | 49.28 | 51.78 | 46.78 | 44.28 | 41.78 |
| (additional) | | | | | |
| Croscarmellose Sodium | 4.05 | 4.05 | 4.05 | 4.05 | 4.05 |
| (additional) | | | | | |
| Magnesium stearate | 0.68 | 0.68 | 0.68 | 0.68 | 0.68 |
| (additional) | | | | | |

Extended release layer:

| Brivaracetam | | 75.00 | 67.50 | 82.50 | 90.00 | 97.50 |
|---|---|---|---|---|---|---|
| Extended release | Hypromellose | 188.80 | 188.80 | 188.80 | 188.80 | 188.80 |
| skeleton material | K200M | | | | | |

| | | | | | |
|---|---|---|---|---|---|
| Magnesium stearate | 0.79 | 0.79 | 0.79 | 0.79 | 0.79 |
| Silicified microcrystalline | 74.62 | 82.12 | 67.12 | 59.62 | 52.12 |
| cellulose (additional) | | | | | |
| Magnesium stearate | 0.79 | 0.79 | 0.79 | 0.79 | 0.79 |
| (additional) | | | | | |

Preparation: 1. Extended release layer: 1) Brivaracetam through a 20-mesh sieve was mixed with the hypromellose K200M (trade name: Benecel™ K200M Pharm) for 30 min, and the magnesium stearate was added to the mixing bucket for 5 min, and the mixture was then dried granulation; 2) the sized granules were mixed with silicified microcrystalline cellulose (additional) for 30 min, magnesium stearate (additional) was added to the mixing bucket and the mixture was continued to mix for 5 min, as an extended release layer particle;
2. Immediate release layer: 1) Brivaracetam through a 20-mesh sieve was mixed with the lactose monohydrate and croscarmellose sodium for 30 min, and the magnesium stearate was added to the mixing bucket for 5 min, and the mixture was then dried granulation; 2) the sized granules were mixed with Anhydrous lactose (additional) and croscarmellose sodium (additional) for 30 min, magnesium stearate (additional) was added to the mixing bucket and the mixture was continued to mix for 5 min, as an immediate release layer particle;
3. The two parts of the pellet were pressed into a bilayer tablet;
4. Opadry 85F coating solution was configured to coat the above-mentioned immediate release extended release bilayer tablet core, and the percentage of coating weight gain in the tablet core was 2%.

According to USP<711> the volume of the medium was 900±9 ml, the temperature of the medium was 37.0±0.5° C., and the rotational speed of the paddle method was 50 rpm or 100 rpm. The dissolution characteristics of tablets F23, F61, F62, F63 and F64 in vitro pH 6.8 dissolution medium were determined at 16 h intervals.

TABLE 15
Dissolution (%) (n = 3) of example 12 and reference listed drug in pH 6.8 medium

| Group - | IR | ER | General | | | | | | | | |
|---|---|---|---|---|---|---|---|---|---|---|---|
| Dissolution | Specifications/ | Specifications/ | Specifications/ | | | | | | | | |
| speed | mg | mg | mg | 15 min | 30 min | 1 h | 2 h | 4 h | 8 h | 12 h | 16 h |
| F23-50 rpm | 25.0 | 75.0 | 100.0 | 28 | 32 | 39 | 50 | 64 | 78 | 91 | 96 |
| F23-100 rpm | | | | 30 | 32 | 40 | 51 | 66 | 80 | 93 | 98 |
| F61-50 rpm | 22.5 | 67.5 | 90.0 | 29 | 33 | 42 | 53 | 66 | 79 | 93 | 97 |
| F61-100 rpm | | | | 32 | 34 | 42 | 53 | 67 | 80 | 94 | 98 |
| F62-50 rpm | 27.5 | 82.5 | 110.0 | 28 | 31 | 38 | 50 | 64 | 78 | 91 | 97 |
| F62-100 rpm | | | | 29 | 30 | 39 | 51 | 65 | 80 | 93 | 98 |
| F63-50 rpm | 30.0 | 90.0 | 120.0 | 27 | 30 | 37 | 49 | 62 | 75 | 90 | 96 |
| F63-100 rpm | | | | 29 | 32 | 39 | 50 | 64 | 77 | 92 | 97 |
| F64-50 rpm | 32.5 | 97.5 | 130.0 | 27 | 30 | 38 | 50 | 63 | 76 | 91 | 95 |
| F64-100 rpm | | | | 29 | 31 | 40 | 51 | 64 | 78 | 93 | 97 |
| Reference | / | / | / | 101 | — | — | — | — | — | — | — |
| listed drug | | | | | | | | | | | |
| (trade name: | | | | | | | | | | | |
| BRIVIACT) | | | | | | | | | | | |

The results of the tablets F23, F61, F62, F63 and F64 prepared in example 12 show that when the specification is 90 mg-130 mg (specification=IR specification+ER specification), the brivaracetam immediate release extended release bilayer tablet prepared by the present invention satisfied the mass of the active ingredient released cumulatively in the buffer medium of pH 6.8 is not less than 20% of the total mass of the active ingredient mass at 15 minutes of dissolution, the mass of the active ingredient cumulatively released in 1 hour of dissolution is 30%-50% of the total mass of the active ingredient, and the mass of the active ingredient cumulatively released after 4 hours of dissolution is 50%-70% of the total mass of the active ingredient, the mass of the active ingredient cumulatively released after 8 hours of dissolution is 70%-85% of the total mass of the active ingredient, and the mass of the active ingredient cumulatively released after 16 hours of dissolution is not less than 90% of the total mass of the active ingredient. Because the 130 mg specification has a higher exposure compared to RLD, the preferred specification is 90 mg to 120 mg.

### Example 13: Pharmacokinetic Studies in Beagle Dogs

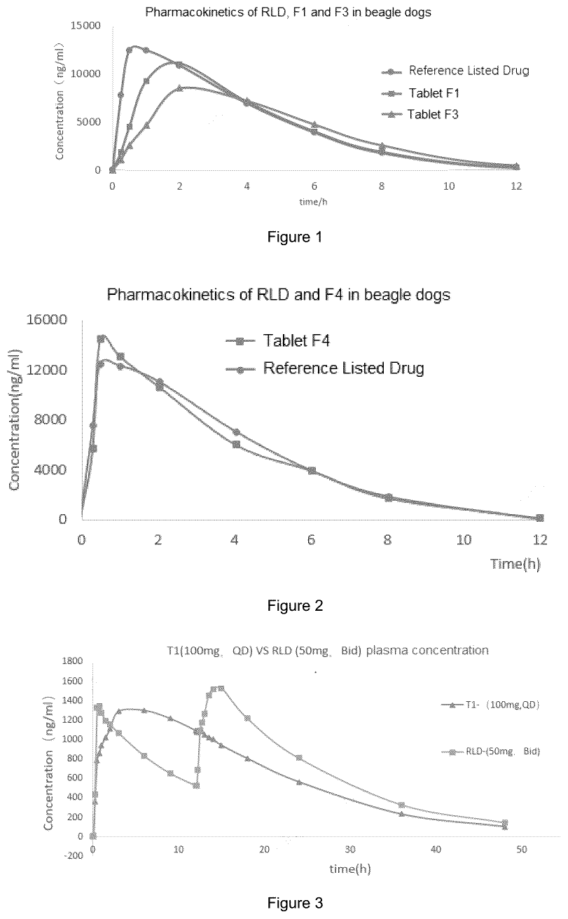

Dosing regimen: Three preparations and three crosses were used (18 healthy Beagle dogs, half male and half female, were divided into 3 groups, 6 in each group, administered on an empty stomach), two of the groups were given tablet F1 and tablet F3 in comparative example 1 once a day; and the other group was given a reference listed drug of 100 mg immediate release preparation (trade name: BRIVIACT) once a day. The results are shown in Table 16 and FIG. 1.

Table 16 listed the pharmacokinetic data for tablet F1, tablet F3 and reference listed drug (trade name: BRIVIACT) in Beagle dogs in comparative example 1.

TABLE 16
Pharmacokinetics of tablet F1, tablet F3 and reference
listed drug in Beagle dogs in comparative example 1

| | | | Reference |
|---|---|---|---|
| PK parameter | Tablet F1 | Tablet F3 | listed drug |
| tmax (ng/ml) | 1 | 2 | 0.5 |
| Cmax (ng/ml) | 11700 ± 1540 | 8910 ± 2780 | 13900 ± 1370 |
| AUC0-t (ng · h/ml) | 53900 ± 9550 | 50400 ± 7910 | 59300 ± 8040 |

Pharmacokinetic studies in Beagle dogs showed that the peak time of tablets F1 and tablets F3 prepared with reference to patent CN102046153 was delayed compared with that of the reference listed drug (trade name: BRIVIACT), showing obvious extended release characteristics. The results of the experiment showed that the extended release tablets alone (without the immediate release layer) did not work quickly in vivo.

### Example 14: Pharmacokinetic Studies in Beagle Dogs

Dosing regimen: Two preparations and two crosses were used (6 healthy Beagle dogs, half male and half female, were divided into 2 groups, 3 in each group, administered on an empty stomach), one of the groups was given tablet F4 in comparative example 1 once a day; and the other group was given a reference listed drug of 100 mg immediate release preparation (trade name: BRIVIACT) once a day. The results are shown in Table 17 and FIG. 2.

Table 17 listed the pharmacokinetic data for tablet F4 and reference listed drug (trade name: BRIVIACT) in Beagle dogs in comparative example 1.

TABLE 17
Pharmacokinetics of tablet F4 and reference listed
drug in Beagle dogs in comparative example 1

| | | Reference |
|---|---|---|
| PK parameter | Tablet F4 | listed drug |
| tmax (ng/ml) | 0.6 | 0.5 |
| Cmax (ng/ml) | 12930 ± 2470 | 13180 ± 1250 |
| AUC0-t (ng · h/ml) | 58400 ± 6190 | 59300 ± 7940 |

The pharmacokinetic study in Beagle dogs showed that the tablet F4 prepared with reference to patent CN102046153 could quickly take effect in Beagle dogs by adjusting the viscosity and proportion of the extended release material, but it was easy to be released suddenly in animals, and the data showed that the extended release effect was not achieved.

### Example 15: Pharmacokinetic Study of the Human Body

Dosing regimen: Randomized double crossover was used for the experiment, the tablet F23 (100 mg) was administered on an empty stomach, once a day, and the other group was given a reference listed drug, the reference listed drug was a 50 mg immediate release preparation (trade name: BRIVIACT), administered twice a day. Twenty-four healthy subjects were enrolled, randomized, administered on an empty stomach, and dosing regimen:
Group 1: Brivaracetam immediate release reference listed drug (trade name: BRIVIACT), specification 50 mg, administered twice a day;
Group 2: T1 (F23), extended release preparation, specification 100 mg, administered once a day;

Blood was collected at the following times after dosing for blood concentration analysis:
0.083 h, 0.25 h, 0.5 h, 0.75 h, 1.0 h, 1.5 h, 2.0 h, 3.0 h, 6.0 h, 9.0 h, 12.0 h, 12.083 h, 12.25 h, 12.5 h, 12.75 h, 13.0 h, 13.5 h, 14.0 h, 15.0 h, 18.0 h, 24.0 h, 36.0 h, 48.0 h. The results are shown in Table 18 and FIG. 3.

TABLE 18
Pharmacokinetic data of the tablet F23 reference
listed drug (trade name: BRIVIACT) in human body

| | T1 (tablet F23) | Reference listed drug | |
|---|---|---|---|
| | Mean ± SD | Mean ± SD | |
| PK parameter | (N = 23) | (N = 23) | Ratio |
| Tmax (h) | 4.761 ± 2.368 | 11.283 ± 5.127 | N/A |
| Cmax (ng/ml) | 1355.979 ± 176.2461 | 1910.008 ± 356.875 | 70.99% |
| AUC₀₋ₜ | 30657.709 ± 5339.238 | 33899.151 ± 4672.171 | 90.44% |
| (ng · h/ml) | | | |
| AUC_{0-inf} | 32194.343 ± 5907.247 | 36045.531 ± 5498.043 | 89.31% |
| (ng · h/ml) | | | |

Note:
Tmax and Cmax:
The Tmax of T1 (extended release tablets prescription by F23) was similar among individuals, and the mean and curve of Cmax were similar in the range of 3-6 h.
There was a large difference in Tmax among individuals with RLD, with some volunteers reaching Cmax after the first dose and some volunteers reaching Cmax after the second dose, resulting in a large difference in Tmax (0.5-15.0 h), and the statistical Cmax data (average of maximum plasma concentration data) was higher than that of the average plasma concentration (the average plasma concentration of different volunteers at the same time point).
*Subsequent modeling simulations were used to calculate the average plasma concentration, so the Cmax data of RLD was 1530.717 ng/ml at 15 h concentration.

The pharmacokinetic study in human body shows that the product prepared by the invention has good pharmacokinetic properties, has a faster onset of action when administered on an empty stomach, has an obvious slow-release effect in human body, and 24 h blood drug concentration is greater than 572 ng/ml.

The average blood concentration-time curve of tablet F23 was analyzed by Wanger-Nelson method, and the absorption percentage (Fabs %)-time curve of brioxetan in vivo was obtained. Further, through the in vivo and in vitro correlation study, the inventors found that the in vitro dissolution method had better in vivo and in vitro correlation, established in vivo and in vitro correlation, and conducted simulation verification of the data. Correlation was obtained by modeling.

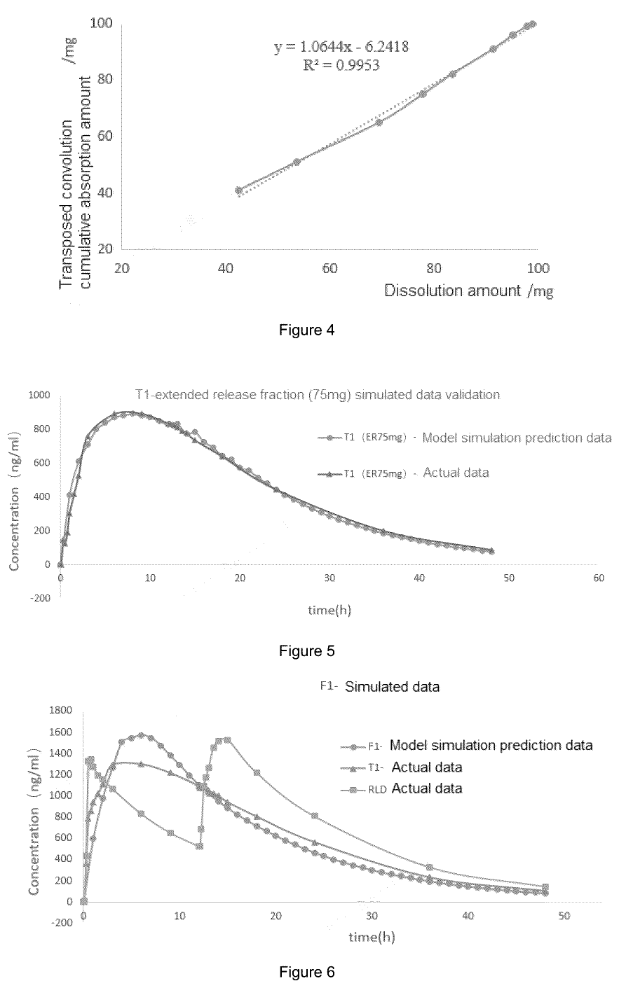

According to the Wagner-Nelson method, the average blood concentration-time curve of the drug was deconvolution technology to obtain the intracorporeal absorption percentage-time curve (see FIG. 4). By drawing the intracorporeal drug absorption percentage-extracorporeal drug release percentage-curve, an intracorporeal and intracorporeal correlation model (IVIVC) can be established. A good linear relationship was obtained with the correlation coefficient greater than 0.95.

### Example 16: Validation of Human PK Model (T1 Extended Release Part)

T1 (F23) prescription was composed of 25 mg immediate release layer and 75 mg extended release layer, and the pharmacokinetics of brivaracetam tablets were linear. The blood concentration of 25 mg was converted by linear method, and the concentration of 25 mg rapid release was deducted from the blood concentration of T1 to obtain the blood concentration data of a single sustained release partial time in T1 prescription. Through the established absorption modeling, IVIVC simulation was performed on the extended-release fraction of T1, and the in vivo absorption fraction was obtained, and the pharmacokinetic data were predicted by integral convolution according to the drug release rate/in vivo absorption rate, and the results are shown in Table 19 and FIG. 5.

TABLE 19
In vivo PK parameters in the extended release portion
of T1 were verified by IVIVC model simulation

| | Cmax | AUC0-t |
|---|---|---|
| Group | (ng/mL) | (hr*ng/mL) |
| T1-ER75 mg (N = 23) | 896.398 | 21968.934 |
| Actual converted data | | |
| T1-IVIVC model simulation | 891.544 | 21908.994 |
| data | | |
| Simulation/actuality (ratio) | 99.46% | 99.73% |

It has been verified that the in vivo and in vitro correlation model established by T1 bilayer can simulate the in vivo data of the single slow-release part of T1. The predicted data simulated by the model has a good correlation with the actual data, and the in vivo pharmacokinetic characteristics of products with different in vitro release rates can be simulated by the established in vivo and in vitro correlation data.

### Example 17: Prediction of In Vivo PK Data for Comparative Example F1 Prescriptions

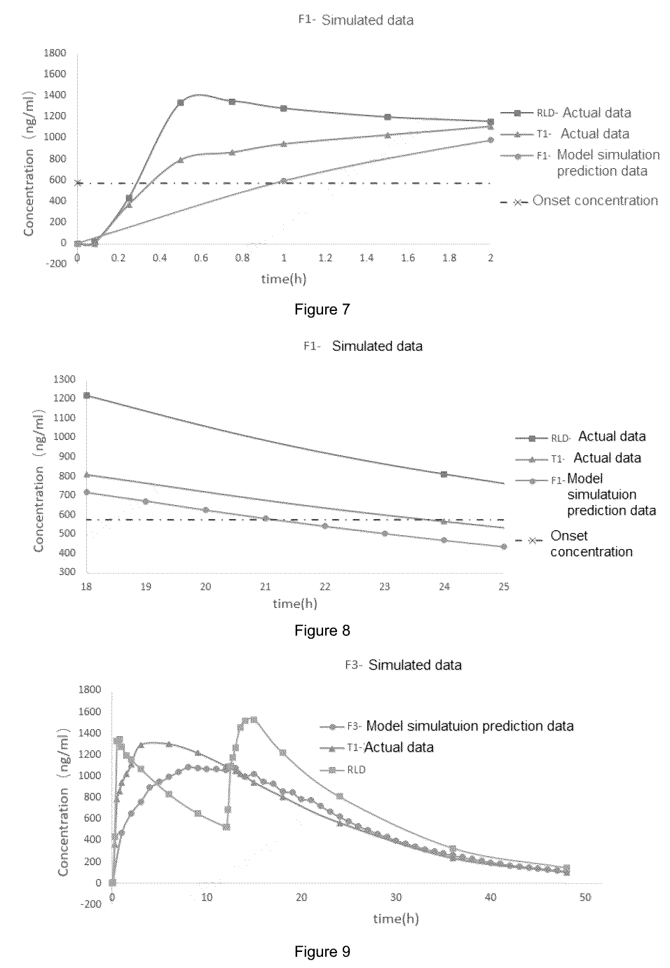

Through in vitro and in vivo correlation studies, the validated model was obtained, and the dissolution data of F1 prescription were integrated and convoluted to predict in vivo data, and the results are shown in Table 20 and FIGS. 6, 7, and 8.

TABLE 20
In vivo PK parameters for F1 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1575.097 | 29106.911 | About 1.0 h | 21 h |
| the F1-IVIVC | | | | |
| model | | | | |
| Simulation/ | 102.90% | 85.86% | N/A | N/A |
| actuality (ratio) | | | | |

In vivo pharmacokinetic curve simulation of F1 prescription was conducted, and it was found that for single-phase extended release preparations, the early release speed was fast, and the early release speed was still slow, resulting in a prolonged onset time (1 h); while the intermediate release speed was fast, resulting in a relatively high Cmax rush of the extended release preparations, resulting in a decrease in the amount of drug released in the later stage. The effective treatment concentration (572 ng/ml) was maintained for only 21 h. The clinical disadvantage of this single-phase extended release formulation is its slow onset and short duration of effective concentration. For patients with acute seizures and long-term medication, there is a “vacuum” period of treatment, resulting in low clinical efficacy of extended release preparations, resulting in serious consequences of seizures.

### Example 18: Prediction of In Vivo PK Data for Comparative Example F3 Prescription

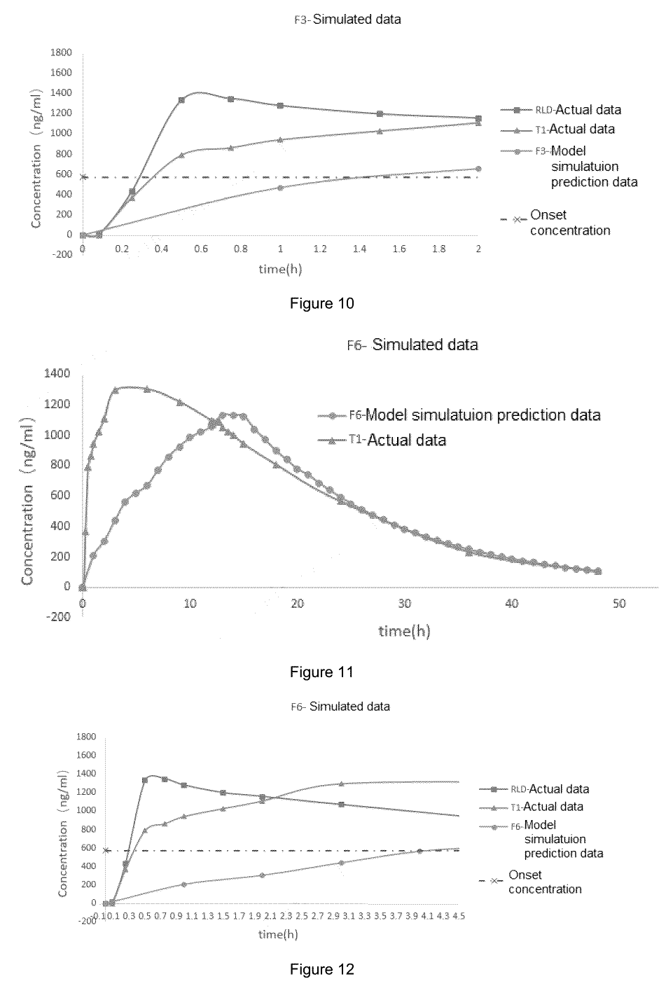

Through in vitro and in vivo correlation studies, the validated model was obtained, and the dissolution data of F3 prescription were integrated and convoluted to predict in vivo data, and the results are shown in Table 21 and FIGS. 9 and 10.

TABLE 21
In vivo PK parameters for F3 prescription
were simulated by the IVIVC model

| | | | Time to first reach |
|---|---|---|---|
| | Cmax | AUC0-t | onset concentration |
| Group | (ng/mL) | (hr*ng/mL) | of 572 ng/ml/(h) |
| RLD actual data | 1530.717 | 33899.15 | About 0.3 h |
| Simulation data of the | 1087.906 | 28148.19 | About 1.4 h |
| F3-IVIVC model | | | |
| Simulation/actuality | 71.07% | 83.04% | N/A |
| (ratio) | | | |

Through in vivo pharmacokinetic curve simulation of F3 prescription, it was found that the Cmax of a single extended release formulation was significantly lower than that of RLD, and the time to reach the effective concentration (572 ng/ml) was also significantly longer than that of the reference listed drug. As a result, the effective time after oral administration of a single extended release formulation was significantly prolonged, which may delay the treatment of the disease. There are significant clinical risks. The inventors were pleasantly surprised to find that the use of biphasic release preparations can quickly reach the onset of concentration, quickly alleviate patients' symptoms, and solve possible clinical risks, which is of clinical significance.

### Example 19: Prediction of In Vivo PK Data for Comparative Example F6 Prescription

Through in vitro and in vivo correlation studies, the validated model was obtained, and the dissolution data of F6 prescription were integrated and convoluted to predict in vivo data, and the results are shown in Table 22 and FIGS. 11 and 12.

TABLE 22
In vivo PK parameters for F6 prescription
were simulated by the IVIVC model

| | | | Time to first reach |
|---|---|---|---|
| | Cmax | AUC0-t | onset concentration |
| Group | (ng/mL) | (hr*ng/mL) | of 572 ng/ml/(h) |
| RLD actual data | 1530.717 | 33899.15 | About 0.3 h |
| Simulation data of the | 1135.535 | 25446.19 | About 4.0 h |
| F6-IVIVC model | | | |
| Simulation/actuality | 74.18% | 75.06% | N/A |
| (ratio) | | | |

The in vivo pharmacokinetic curve simulation of F6 prescription showed that Cmax was lower than that of RLD using a single membrane controlled extended release preparation, and the time to reach the effective concentration (572 ng/ml) was also significantly longer than that of the reference listed drug, so that the onset time of oral administration of a single extended release preparation was significantly extended (from 0.3 h to 4 h of RLD). Treatment of the disease may be delayed, and there are significant clinical concerns for patients who may have an acute episode. The inventors were pleasantly surprised to find that the use of biphasic release preparations can quickly reach the onset of concentration, quickly alleviate patients' symptoms, and solve possible clinical risks, which is of clinical significance.

### Example 20: Prediction of In Vivo PK Data for Comparative Example F4 Prescription

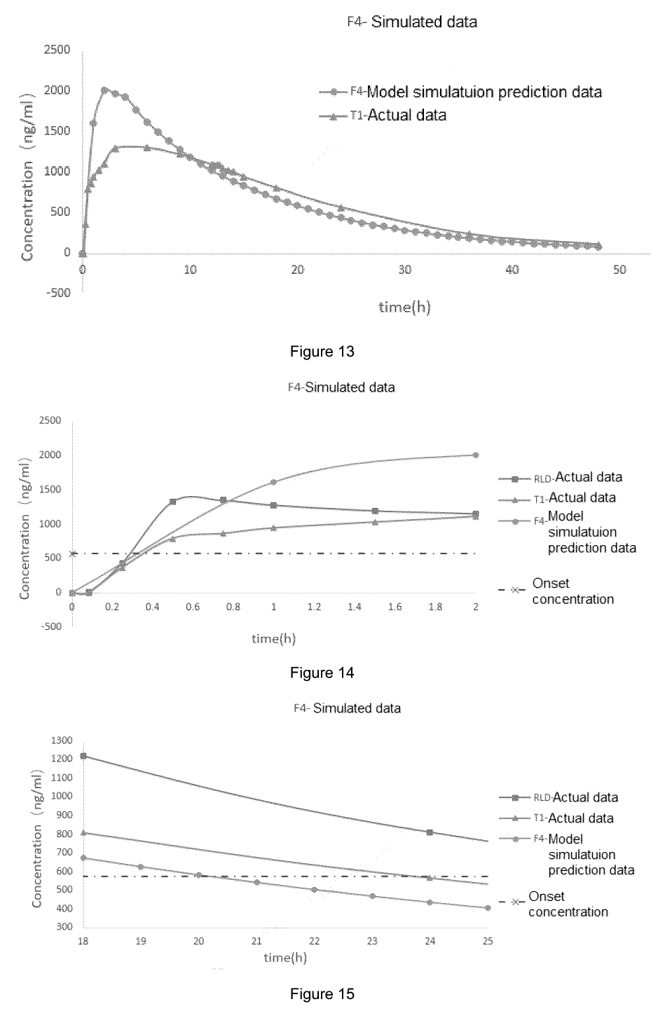

Through in vitro and in vivo correlation studies, the validated model was obtained, and the dissolution data of F4 prescription were integrated and convoluted to predict in vivo data, and the results are shown in Table 23 and FIGS. 13, 14 and 15.

TABLE 23
In vivo PK parameters for F4 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 2004.311 | 31123.84 | About 0.3 h | 20 h |
| the F4-IVIVC | | | | |
| model | | | | |
| Simulation/ | 130.94% | 91.81% | N/A | N/A |
| actuality (ratio) | | | | |

Through in vivo pharmacokinetic curve simulation of F4 prescription, it was found that the Cmax of the extended release formulation with a faster release rate before 1 h was higher than that of RLD, and there was a certain risk of sudden release. After continuous administration, the accumulation of Cmax may bring safety risks. In terms of onset time, F4 reached the effective concentration (572 ng/ml) faster, which was similar to the reference listed drug. However, the release rate is too fast and not stable enough, resulting in the effective concentration maintenance time of only 20 h, which is obviously shorter than the reference preparation of the biphasic preparation of the invention, resulting in the curative effect blank period in the clinical use process, and bringing great risks to patients.

### Example 21: Prediction of In Vivo PK Data for Comparative Example F7 Prescription

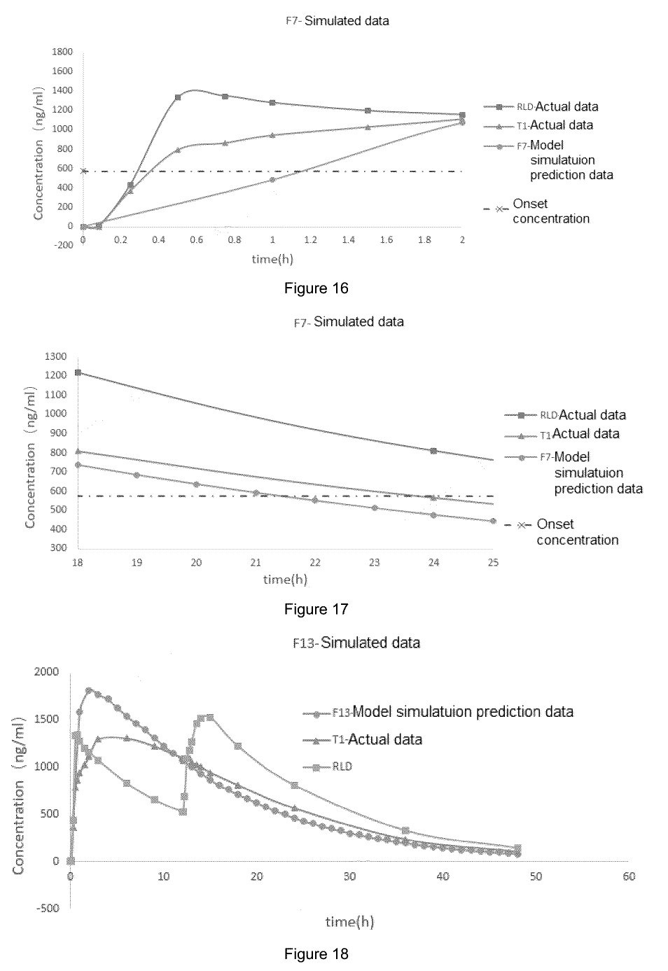

Through in vitro and in vivo correlation studies, the validated model was obtained, and the dissolution data of F7 prescription were integrated and convoluted to predict in vivo data, and the results are shown in Table 24 and FIGS. 16 and 17.

TABLE 24
In vivo PK parameters for F7 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1557.884 | 28591.65 | About 1.2 h | 21 h |
| the F7-IVIVC | | | | |
| model | | | | |
| Simulation/ | 101.77% | 84.34% | N/A | N/A |
| actuality (ratio) | | | | |

The in vivo pharmacokinetic curve simulation of F7 prescription showed that the release rate was slow before 1 h, resulting in a prolonged onset time. The time for F7 to reach the effective concentration (572 ng/ml) was 1.2 h, which was significantly slower than the reference listed drug, bringing certain risks to clinical treatment. At the same time, the mid-term release rate of the drug is too fast, and its release rate is too fast, and it is not stable enough, resulting in the time of maintaining the effective concentration of only 21 h, which is significantly shorter than the biphasic preparation of the present invention of the reference listed drug, so that there is a blank period of efficacy in the process of clinical use, and it brings great risk to patients.

### Example 22: Prediction of In Vivo PK Data for Comparative Example F13 Prescription

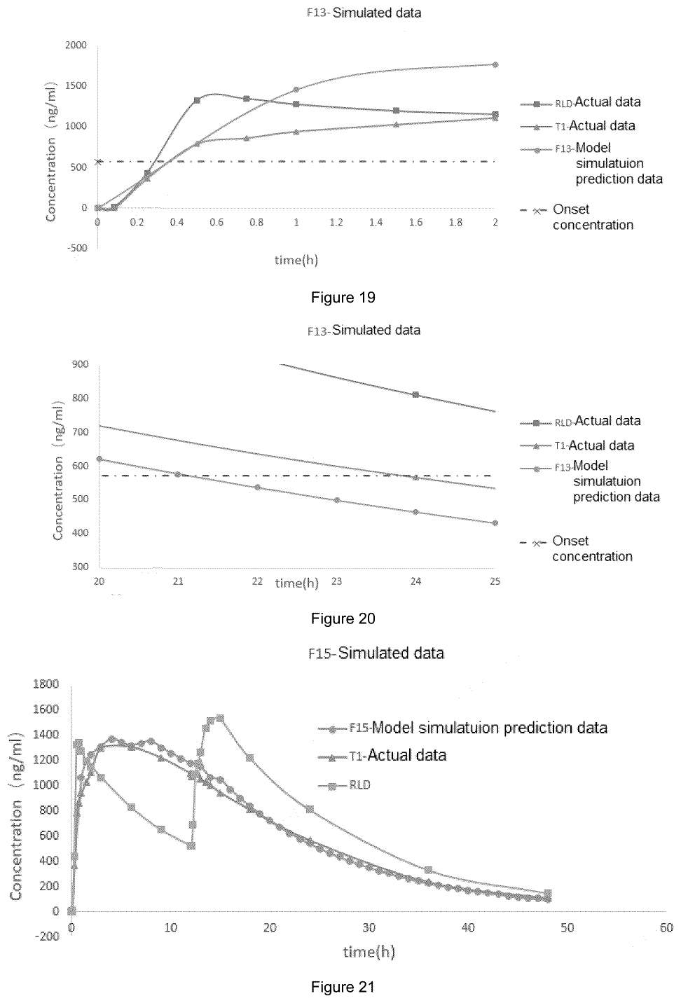

Through in vitro and in vivo correlation studies, the validated model was obtained, and the dissolution data of F13 prescription were integrated and convoluted to predict in vivo data, and the results are shown in Table 25 and FIGS. 18, 19 and 20.

TABLE 25
In vivo PK parameters for F13 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1809.758 | 31145.96 | About 0.3 h | 21 h |
| the F13-IVIVC | | | | |
| model | | | | |
| Simulation/ | 118.23% | 91.88% | N/A | N/A |
| actuality (ratio) | | | | |

The in vivo pharmacokinetic curve simulation of F13 prescription showed that the use of K4M as the extended release skeleton material, because of the high water solubility of the API, HPMC K4M still could not well control the early burst release, because the early release rate was too fast, there was a significant risk of sudden release, Cmax was higher than RLD, and after multiple doses, the accumulated blood drug concentration would be higher, resulting in clinical safety risks. At the same time, because of the sudden release, the blood concentration was not stable enough, and the time to maintain a stable blood concentration was only 21 hours, which made the efficacy blank period in the process of clinical use, which brought great risks to patients, and at the same time, the peak and valley ratio were relatively large, and the blood concentration fluctuated greatly.

### Example 23: Prediction of In Vivo PK Data for Comparative Example F15 Prescriptions

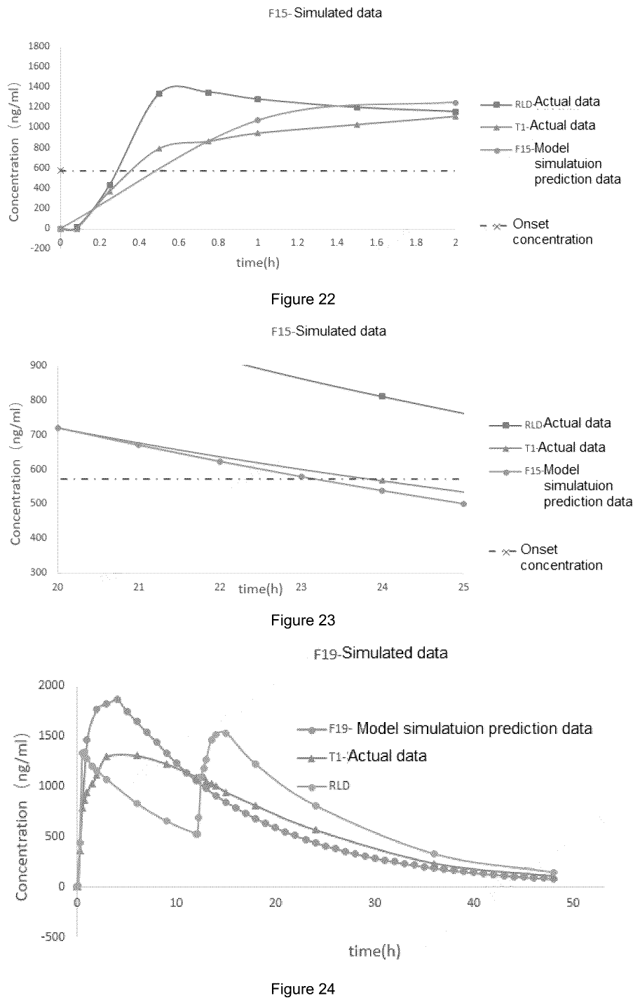

Through in vitro and in vivo correlation studies, the validated model was obtained, and the dissolution data of F15 prescription were integrated and convoluted to predict in vivo data, and the results are shown in Table 26 and FIGS. 21, 22, and 23.

TABLE 26
In vivo PK parameters for F15 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1373.332 | 31045.25 | About 0.5 h | 23 h |
| the F15-IVIVC | | | | |
| model | | | | |
| Simulation/ | 89.72% | 91.58% | N/A | N/A |
| actuality (ratio) | | | | |

The in vivo pharmacokinetic curve simulation of F15 prescription showed that K100M was used as the extended release skeleton material, the API was highly water-soluble, and the sudden release could also be well controlled, and the immediate release layer in the bilayer tablet could be quickly released to the onset concentration (0.5 h), and the effective concentration was maintained for 23 h, and the extended release effect was good.

### Example 24: Prediction of In Vivo PK Data for Comparative Example F19 Prescriptions

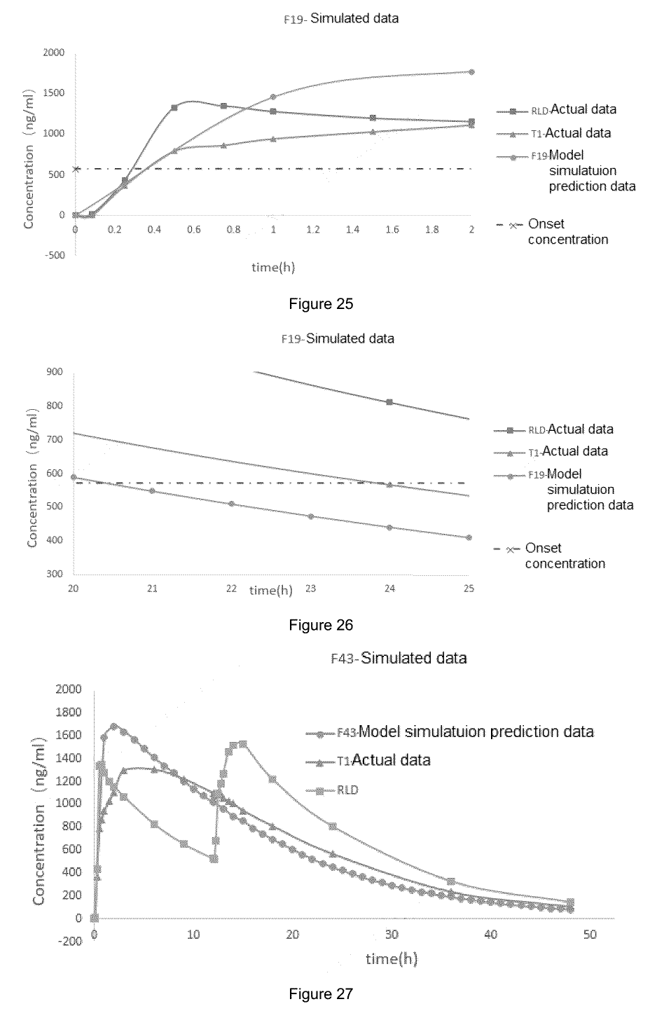

Through in vitro and in vivo correlation studies, the validated model was obtained, and the dissolution data of F19 prescription were integrated and convoluted to predict in vivo data, and the results are shown in Table 27 and FIGS. 24, 25, and 26.

TABLE 27
In vivo PK parameters for F19 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1859.903 | 30942.13 | About 0.3 h | 20.5 h |
| the F19-IVIVC | | | | |
| model | | | | |
| Simulation/ | 121.51% | 91.28% | N/A | N/A |
| actuality (ratio) | | | | |

The in vivo pharmacokinetic curve simulation of F19 prescription showed that the early release rate of K100LV was too fast, and there was a significant risk of sudden release, Cmax was higher than RLD, and after multiple doses, the accumulated blood drug concentration would be higher, resulting in clinical safety risks, and at the same time, because of the sudden release, the blood concentration was not stable enough, and the blood concentration time to maintain stability was only 20.5 h, which made the efficacy blank period in the clinical use process, which brought great risks to patients, and the peak and valley fluctuations were also relatively large.

### Example 25: Prediction of In Vivo PK Data for Comparative Example F43 Prescriptions

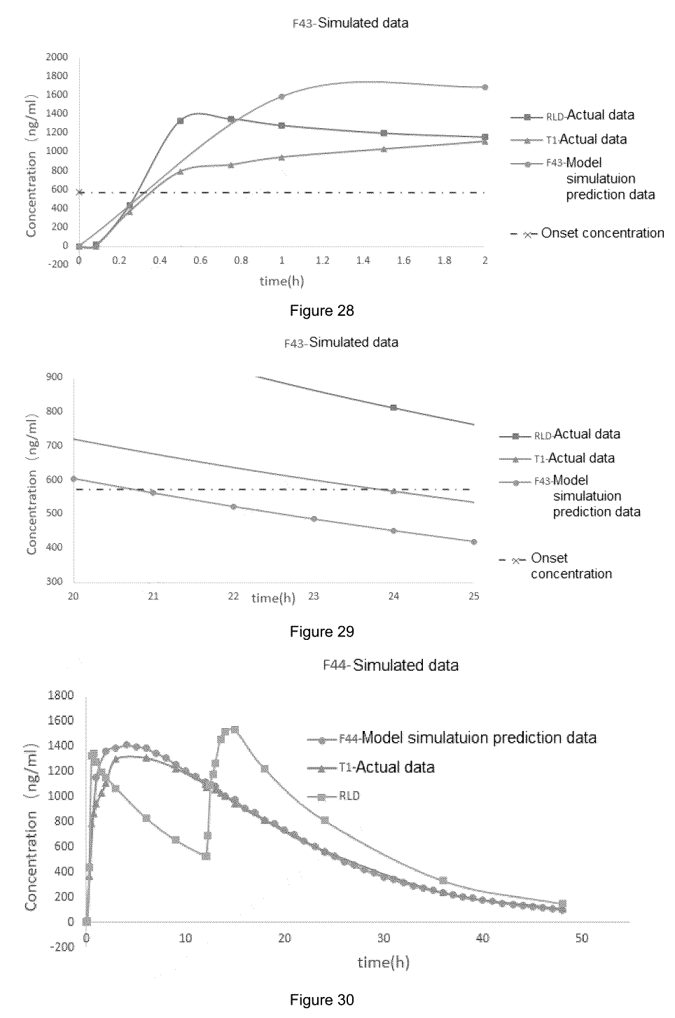

Through in vitro and in vivo correlation studies, the validated model was obtained, and the dissolution data of F43 prescription were integrated and convoluted to predict in vivo data, and the results are shown in Table 28 and FIGS. 27, 28, and 29.

TABLE 28
In vivo PK parameters for F43 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1682.048 | 32613.28 | About 0.3 h | 21 h |
| the F43-IVIVC | | | | |
| model | | | | |
| Simulation/ | 109.89% | 96.21% | N/A | N/A |
| actuality (ratio) | | | | |

The in vivo pharmacokinetic curve simulation of F43 prescription showed that K200M was used as the extended release skeleton material, and the proportion of immediate release layer was as high as 50%, Cmax was higher than RLD, after multiple doses, the accumulated blood drug concentration will be higher, resulting in clinical safety risks, and at the same time, because of the sudden release, the blood concentration was not stable enough, and the blood concentration time to maintain stability was only 21 h, which made the clinical use process appear in the efficacy blank period, which brought great risk to the patient, and the peak and valley fluctuations were also relatively large, and the fluctuation of blood drug concentration will bring more clinical risks.

### Example 26: Prediction of In Vivo PK Data for Comparative Example F44 Prescriptions

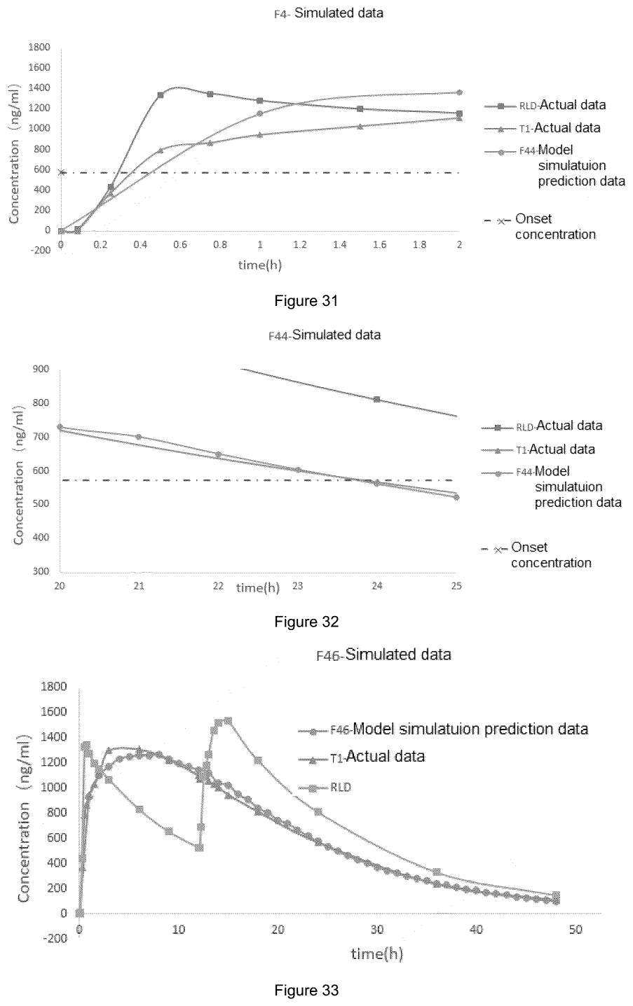

Through in vitro and in vivo correlation studies, the validated model was obtained, and the dissolution data of F44 prescription were integrated and convoluted to predict in vivo data, and the results are shown in Table 29 and FIGS. 30, 31, and 32.

TABLE 29
In vivo PK parameters for F44 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1408.444 | 31232.86 | About 0.4 h | 24 h |
| the F44-IVIVC | | | | |
| model | | | | |
| Simulation/ | 92.01% | 92.13% | N/A | N/A |
| actuality (ratio) | | | | |

The in vivo pharmacokinetic curve simulation of F44 prescription found that when K200M was used as the extended release skeleton material, the proportion of the immediate release layer was as high as 33%, because the high-viscosity extended release material could well control the early release, Cmax was close to the reference listed drug, there was no clinical risk, and at the same time, it could achieve rapid onset and long-term (24 h) maintenance of concentration, and the blood concentration fluctuation was small, which was an excellent extended release preparation.

### Example 27: Prediction of In Vivo PK Data for Comparative Example F46 Prescriptions

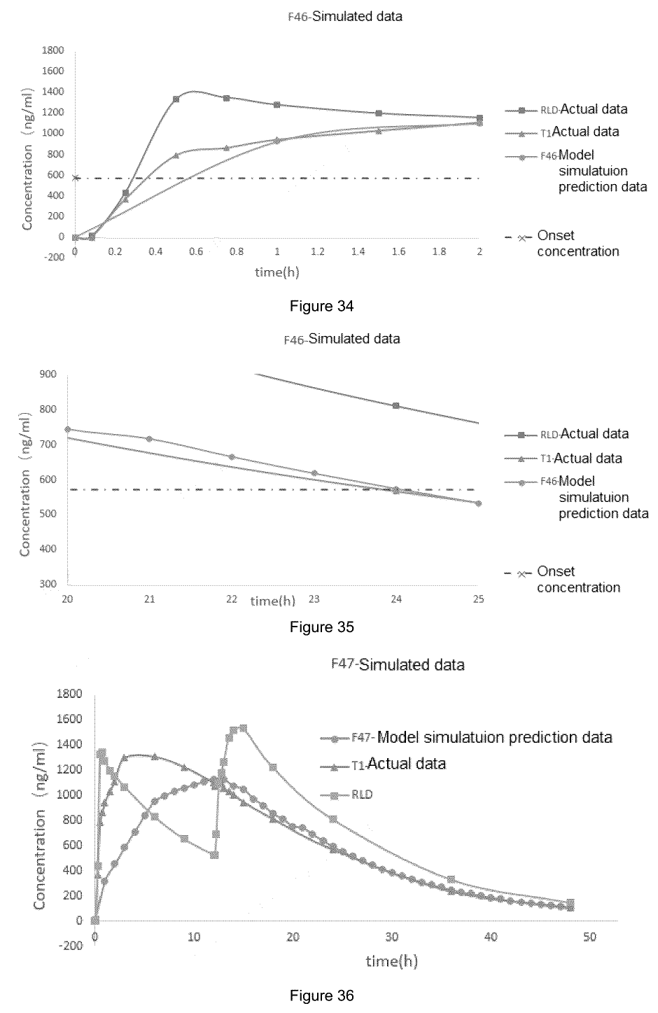

Through in vitro and in vivo correlation studies, the validated model was obtained, and the dissolution data of F46 prescription were integrated and convoluted to predict in vivo data, and the results are shown in Table 30 and FIGS. 33, 34, and 35.

TABLE 30
In vivo PK parameters for F46 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1261.646 | 30396.29 | About 0.6 h | 24 h |
| the F46-IVIVC | | | | |
| model | | | | |
| Simulation/actuality | 82.42% | 89.67% | N/A | N/A |
| (ratio) | | | | |

The in vivo pharmacokinetic curve simulation of F46 prescription showed that when K200M was used as the extended release skeleton material, and the proportion of the immediate release layer was 16%, the immediate release part in the early stage was released quickly, and the effective concentration could be quickly reached, and the Cmax was close to the reference listed drug, with no clinical risk, and at the same time, it could achieve rapid onset and long-term (24 h) maintenance of concentration, and the blood concentration fluctuation was small, which was an excellent extended release preparation.

### Example 28: Prediction of In Vivo PK Data for Comparative Example F47 Prescriptions

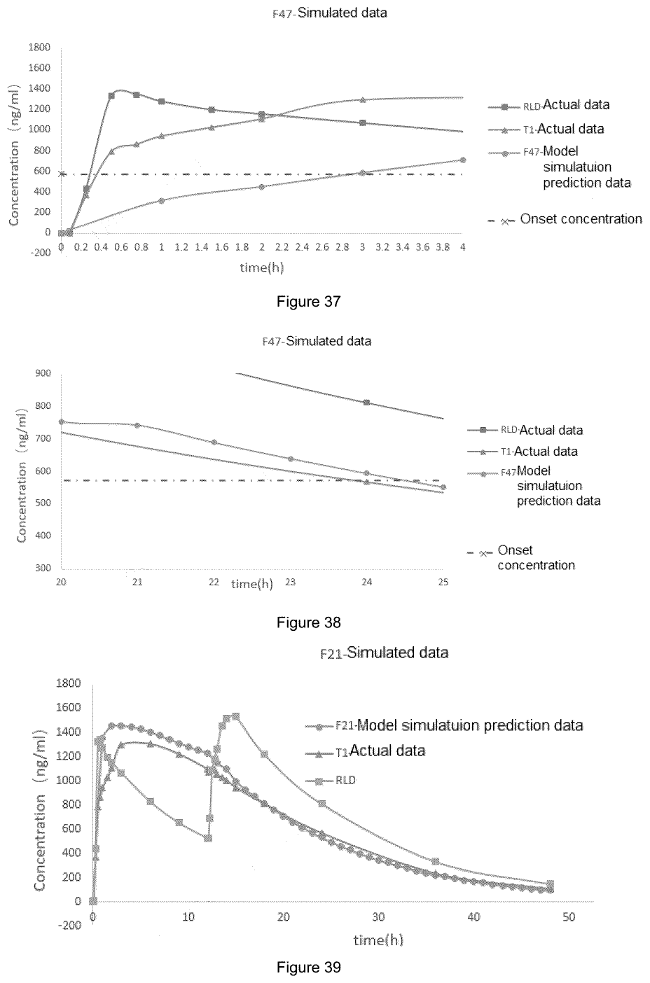

Through in vitro and in vivo correlation studies, the validated model was obtained, and the dissolution data of F1 prescription were integrated and convoluted to predict in vivo data, and the results are shown in Table 31 and FIGS. 36, 37, and 38.

TABLE 31
In vivo PK parameters for F47 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1129.043 | 26909.54 | About 3 h | >24 h |
| the F47-IVIVC | | | | |
| model | | | | |
| Simulation/ | 73.76% | 79.38% | N/A | N/A |
| actuality (ratio) | | | | |

The in vivo pharmacokinetic curve simulation of F47 prescription showed that when K200M was used as the extended release skeleton material, the proportion of immediate release layer was 0%, and the early release was slower, resulting in a significant prolongation of the time to reach the effective concentration (3 h), which may bring greater clinical risks to patients. Because a large amount of the drug is released at the end of the digestive tract, the Cmax and AUC are lower than those of the reference preparation, and the efficacy will be different, resulting in ineffective treatment. Single-layer extended release tablets have certain clinical defects, resulting in the risk of clinical safety and efficacy, and the use of biphasic release technology can well control the risk.

### Example 29: Prediction of In Vivo PK Data for Comparative Example F21 Prescriptions

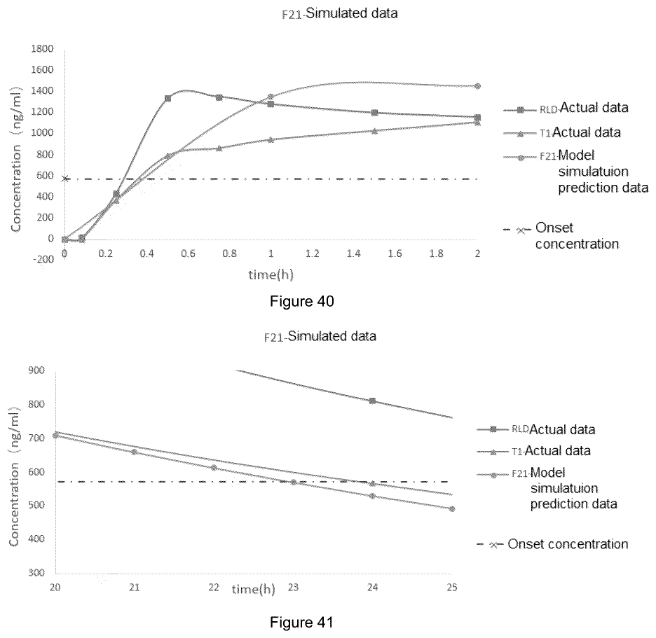

Through in vitro and in vivo correlation studies, the validated model was obtained, and the dissolution data of F21 prescription were integrated and convoluted to predict in vivo data, and the results are shown in Table 32 and FIGS. 39, 40 and 41.

TABLE 32
In vivo PK parameters for F21 prescription
were simulated by the IVIVC model

| | | | | Time to |
|---|---|---|---|---|
| | | | Time to first | maintain |
| | | | reach onset | onset |
| | | | concen- | concen- |
| | | | tration of | tration of |
| | Cmax | AUC0-t | 572 ng/ | 572 ng/ |
| Group | (ng/mL) | (hr*ng/mL) | ml/(h) | ml/(h) |
| RLD actual data | 1530.717 | 33899.15 | About 0.3 h | >24 h |
| Simulation data of | 1547.251 | 31783.68 | About 0.4 h | 23 h |
| the F21-IVIVC | | | | |
| model | | | | |
| Simulation/ | 101.08% | 93.76% | N/A | N/A |
| actuality (ratio) | | | | |

T1 clinical prescription, AUC and RLD equivalent, Cmax was slightly lower, in order to increase Cmax, a lower viscosity extended release material HPMC K15M was simulated to obtain a higher Cmax. The in vivo pharmacokinetic curve simulation of F21 prescription showed that when HPMC KI5M was used as the extended release skeleton material, the proportion of the immediate release layer was 25%, and it could be quickly released in the early stage, quickly reaching the onset concentration (0.4 h), and the time maintained above the effective concentration also reached 23 h. Therefore, F21 and T1 prescriptions were selected for the study of food effects.

### Example 30: Study of the Food Effect of the Product

Combined with the anthropokinetic study of T1 (prescription F23), T1 (prescription F23) was selected for the study of food effect, and in order to improve the fasting Cmax, a double-layer skeleton piece T2 (prescription F21) with lower viscosity HPMC was selected for the study of food effect.

Dosing regimen: randomized, parallel double-cross experiment, in which 12 healthy subjects were enrolled, randomized into 2 groups, 6 subjects in each group, respectively for T1 and T2 food effect studies, fasting administration, dosing regimen:
Group A: T1, the specification was 100 mg, administered once a day, fasting+feeding (N=6);
Group A: T2, the specification was 100 mg, administered once a day, fasting+feeding (N=6);
   In parallel double-cross experiments, blood was collected at the following times after dosing for blood concentration analysis:
0.083 h, 0.25 h, 0.5 h, 0.75 h, 1.0 h, 1.5 h, 2.0 h, 3.0 h, 6.0 h, 9.0 h, 12.0 h, 12.083 h, 12.25 h, 12.5 h, 12.75 h, 13.0 h, 13.5 h, 14.0 h, 15.0 h, 18.0 h, 24.0 h, 36.0 h, 48.0 h.

Referring to FDA. Guidance for Industry: Assessing the Effects of Food on Drugs in INDs and NDAs—Clinical Pharmacology Considerations, 2019. For the study of food effect, it is required that Cmax and AUC are equivalent after fasting and eating, and it can be determined that food has no significant effect on the bioavailability of the drug, and if the ratio value exceeds 80.00-125.00%, it is impossible to be equivalent. Food effect studies on T1 and T2 were carried out, and the results are shown in Tables 33 and 34.

TABLE 33
Pharmacokinetic data in humans for T1 food effects

| | Cmax | AUC0-t | AUC0-inf |
|---|---|---|---|
| Group | (ng/mL) | (hr*ng/mL) | (hr*ng/mL) |
| T1 (N = 6) fasting | 1359.038 | 30800.842 | 32339.120 |
| T1 (N = 6) feeding | 1520.215 | 32190.258 | 33893.782 |
| T1 feeding/fasting (ratio) | 111.86% | 104.51% | 104.81% |

TABLE 34
Pharmacokinetic data in humans for T2 food effects

| | Cmax | AUC0-t | AUC0-inf |
|---|---|---|---|
| Group | (ng/mL) | (hr*ng/mL) | (hr*ng/mL) |
| T2 (N = 6) fasting | 1585.155 | 32021.687 | 33416.576 |
| T2 (N = 6) feeding | 1931.812 | 33192.241 | 34218.684 |
| T2 feeding/fasting (ratio) | 121.87% | 103.66% | 102.40% |

As we all know, after eating, the peristalsis ability of the stomach is intensified, and there is a greater pressure in food squeezing, which often accelerates the wear of the skeleton piece, resulting in the risk of sudden release. The inventors were surprised to find that with the T1 prescription (HPMC K200M skeleton), the product had no significant food effect, and the Cmax increased by 11% and the AUC increased by 5% after eating. T2 prescription (HPMC K15M skeleton), the product did not have a significant food effect, but Cmax increased by nearly 22% and AUC increased by about 2% after ingestion. Comparing T1 and T2 prescriptions in parallel, the inventors found that the Cmax of T2 was higher than that of T1 under fasting conditions, and if T2 feeding and T1 fasting were compared in parallel, the inventors unexpectedly found that the Cmax of T2 feeding was about 42% higher than that of T1 fasting. Combined with in vitro data, the inventors found that the in vitro dissolution above 100 rpm in the paddle method has a high correlation with feeding, and the inventor found that the dissolution of materials below K15M viscosity will be faster at high speeds, which will lead to a significant food effect in the extended release preparation using low-viscosity materials, and after feeding, due to the friction of food, the sudden release of the drug will greatly increase the Cmax and bring the risk of clinical safety. Through a large number of experiments, the inventors were pleasantly surprised to find that when HPMC is used as the skeleton material to prepare biphasic extended release tablets, the viscosity is K15M-K200M, and the good extended release absorption characteristics in vivo can be obtained, and the product is less affected by food.

The method of the present invention has been described by the optimal embodiment, related person can clearly realize and apply the techniques disclosed herein by making some changes, appropriate alterations or combinations to the methods without departing from spirit, principles and scope of the present disclosure. Skilled in the art can learn from this article to properly improve the process parameters to implement the preparation method. Of particular note is that all similar substitutions and modifications to the skilled person is obvious, and they are deemed to be included in the present invention.

1-27. (canceled)

28. A tablet, comprises an extended release layer and an immediate release layer, the extended release layer comprises an active ingredient, an extended release skeleton material and optional other pharmaceutically acceptable adjuvants or carriers, the immediate release layer comprises an active ingredient and other adjuvants or carriers; the active ingredient is brivaracetam or a pharmaceutically acceptable salt thereof, the extended release skeleton material comprises or is hypromellose, the viscosity of the solution obtained by dissolving the hypromellose in water at a concentration of 2 wt % is 13500 mPa·s-280000 mPa·s at 20° C.±0.1° C.

29. The tablet according to claim 28, the hypromellose comprises at least one of hypromellose K15M, hypromellose K100M and hypromellose K200M.

30. The tablet according to claim 28, the extended release skeleton material accounts for 30 wt %-74 wt % or 30.0 wt %-77.5 wt % of the total weight of the extended release layer; or the extended release skeleton material is hypromellose, the hypromellose is hypromellose K15M, the extended release skeleton material accounts for 55.5 wt %-77.5 wt % of the total weight of the extended release layer; or the extended release skeleton material is hypromellose, the hypromellose is hypromellose K100M, the extended release skeleton material accounts for 46.9 wt %-77.5 wt % of the total weight of the extended release layer; or the extended release skeleton material is hypromellose, the hypromellose is hypromellose K200M, the extended release skeleton material accounts for 30.0 wt %-77.5 wt % of the total weight of the extended release layer.

31. The tablet according to claim 28, the extended release skeleton material accounts for 22 wt %-53 wt % or 21.5 wt %-55.5 wt % of the total weight of the extended release layer and the immediate release layer; or the extended release skeleton material is hypromellose, the hypromellose is hypromellose K15M, the extended release skeleton material accounts for 39.7 wt %-55.5 wt % of the total weight of the extended release layer and the immediate release layer; or the extended release skeleton material is hypromellose, the hypromellose is hypromellose K100M, the extended release skeleton material accounts for 33.6 wt %-53.5 wt % of the total weight of the extended release layer and the immediate release layer; or the extended release skeleton material is hypromellose, the hypromellose is hypromellose K200M, the extended release skeleton material accounts for 21.5 wt %-55.5 wt % of the total weight of the extended release layer and the immediate release layer.

32. The tablet according to claim 28, the other pharmaceutically acceptable adjuvants or carriers in the extended release layer comprise at least one of diluents and lubricants; or
the other adjuvants or carriers in the immediate release layer comprise at least one of diluents, disintegrants and lubricants.

33. The tablet according to claim 32, the diluent in the extended release layer accounts for 3 wt %-29 wt % or 0-34.0 wt % of the total mass of the extended release layer and the immediate release layer; or the extended release skeleton material is hypromellose, the hypromellose is hypromellose K15M, and the diluent in the extended release layer accounts for 0-15.7 wt % of the total mass of the extended release layer and the immediate release layer; or the extended release skeleton material is hypromellose, the hypromellose is hypromellose K100M, and the diluent in the extended release layer accounts for 2.1 wt %-21.9 wt % of the total mass of the extended release layer and the immediate release layer; or the extended release skeleton material is hypromellose, the hypromellose is hypromellose K200M, and the diluent in the extended release layer accounts for 0-34.0 wt % of the total mass of the extended release layer and the immediate release layer.

34. The tablet according to claim 32, the diluent in the immediate release layer accounts for 3 wt %-29 wt % or 5.2 wt %-38.7 wt % of the total mass of the extended release layer and the immediate release layer.

35. The tablet according to claim 32, the lubricant in the extended release layer accounts for 0-2.0 wt % or 0.2 wt %-0.5 wt % or 0.3 wt %-0.5 wt % of the total mass of the extended release layer and the immediate release layer.

36. The tablet according to claim 32, the lubricant in the immediate release layer accounts for 0-2.0 wt % or 0.2 wt %-0.5 wt % or 0.3 wt % of the total mass of the extended release layer and the immediate release layer.

37. The tablet according to claim 32, the disintegrant in the immediate release layer accounts for 0-5.0 wt % or 1.5 wt %-2.0 wt % of the total mass of the extended release layer and the immediate release layer.

38. The tablet according to claim 32, the diluent in the immediate release layer and the diluent in the extended release layer independently comprise at least one of the siliconized microcrystalline cellulose, microcrystalline cellulose, sucrose, lactose, lactose monohydrate, dicalcium phosphate, mannitol, dextrin, starch and pregelatinized starch, respectively; or
the lubricant in the immediate release layer and the lubricant in the extended release layer independently comprise at least one of stearic acid, talc, colloidal silicon dioxide, sodium stearic fumarate, magnesium stearate or calcium stearate, respectively; or the disintegrant comprises at least one of the crospovidone, sodium carboxymethyl starch, croscarmellose sodium, low-substituted hydroxypropyl cellulose, etc.

39. The tablet according to claim 28, brivaracetam or pharmacologically acceptable salts thereof in the immediate release layer account for 16.0 wt %-33.0 wt % or 16.0 wt %-25.0 wt % of the total mass of the active ingredient in the extended release layer and the immediate release layer.

40. The tablet according to claim 28, the accumulated release of active ingredients of the tablet in the buffer medium of pH 6.8 in 15 minutes of dissolution not less than 20% of the total mass of active ingredients, the accumulated release of active ingredients for 1 hour of dissolution is 30%-50% of the total mass of active ingredients, the accumulated release of active ingredients for 4 hours of dissolution is 50%-70% of the total mass of active ingredients, the accumulated release of active ingredients for 8 hours of dissolution is 70%-85% of the total mass of active ingredients, and the accumulated release of active ingredients for 16 hours of dissolution is not less than 90% of the total mass of active ingredients.

41. The tablet according to claim 28, the tablet releases the active ingredient in a buffer medium at pH 6.8 for at least 24 hours.

42. The tablet according to claim 28, the active ingredient is calculated in brivaracetam, and the specifications of the active ingredient in the single tablet are 90 mg-120 mg.

43. The tablet according to claim 28, the tablet also comprises a separator layer or coating encapsulating the extended release layer and/or the immediate release layer.

44. The tablet according to claim 43, the separator layer comprises a water-soluble polymer film-forming material and a plasticiser, and optionally comprises an anti-sticking agent or a sun-blocking agent.

45. The tablet according to claim 43, the separator layer or coating account for 2.0 wt %-4.0 wt % of the total mass of the tablet.

46. A preparation method of the tablet according to claim 28, comprising:
(1) granulation: the active ingredient, extended release skeleton material and other pharmaceutically acceptable excipients or carriers are mixed, and the extended release layer particles are obtained by dry granulation; the active ingredient, other excipients or carriers are mixed, and the immediate release layer particles are obtained by dry granulation and (2) compressing tablet: (a) extended release layer particles are pre-pressed, then filled with immediate release layer particles and tablet-compressed, or (b) immediate release layer particles are pre-pressed, then filled with extended release layer particles and tablet-compressed.

47. The preparation method according to claim 46, further comprising wrapping a separator layer or coating after compressing tablet.
